SEQUENCE LISTING (23 sequences)

SEQ ID NO: 1 - protein, 1706 aa, Bordetella pertussis
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 2 - protein, 697 aa, Artificial Sequence
  Adenylate cyclase fragment without His tag
TENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDP

SEQ ID NO: 3 - protein, 698 aa, Artificial Sequence
  Adenylate cyclase fragment without His tag
TENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPG

SEQ ID NO: 4 - protein, 699 aa, Artificial Sequence
  Adenylate cyclase fragment without His tag
TENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGG

SEQ ID NO: 5 - protein, 703 aa, Artificial Sequence
  Adenylate cyclase plus His tag 1
TENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPHHHHHH

SEQ ID NO: 6 - protein, 704 aa, Artificial Sequence
  Adenylate cyclase plus His tag 2
TENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGHHHHHH

SEQ ID NO: 7 - protein, 705 aa, Artificial Sequence
  Adenylate cyclase plus His tag 3
TENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGGHHHHHH

SEQ ID NO: 8 - protein, 726 aa, Artificial Sequence
  Fragment plus NspA signal peptide
MKKALATLIALALPAAALAEGTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGGHHHHHH

SEQ ID NO: 9 - protein, 4 aa, Artificial Sequence
  linker 1
GSGS

SEQ ID NO: 10 - protein, 6 aa, Artificial Sequence
  Linker 2
GSGGGG

SEQ ID NO: 11 - protein, 8 aa, Artificial Sequence
  linker 3
GSGSGGGG

SEQ ID NO: 12 - protein, 8 aa, Artificial Sequence
  His tag 1
GGHHHHHH

SEQ ID NO: 13 - protein, 7 aa, Artificial Sequence
  His tag 2
GHHHHHH

SEQ ID NO: 14 - protein, 6 aa, Artificial Sequence
  His tag 3
HHHHHH

SEQ ID NO: 15 - protein, 399 aa, Artificial Sequence
  AC domain (from residue 1 to residue 400).
QQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQ

SEQ ID NO: 16 - protein, 401 aa, Artificial Sequence
  AC domain (from residue 1 to residue 400) with GS insertion
QQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADGSIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQ

SEQ ID NO: 17 - protein, 134 aa, Artificial Sequence
  AC domain (from residue 360 to residue 493).
DGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNT

SEQ ID NO: 18 - protein, 400 aa, Artificial Sequence
  AC domain (from residue 1 to residue 400).
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQ

SEQ ID NO: 19 - protein, 402 aa, Artificial Sequence
  AC domain (from residue 1 to residue 400) with GS insertion
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADGSIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQ

SEQ ID NO: 20 - protein, 135 aa, Artificial Sequence
  AC domain (from residue 360 to residue 493).
MDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNT

SEQ ID NO: 21 - protein, 410 aa, Artificial Sequence
  AC domain (from residue 1 to residue 400). Cytoplasmic expression of his-tagged (composed of GGHHHHHH sequence) domain
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADGSIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQGGHHHHHH

SEQ ID NO: 22 - protein, 143 aa, Artificial Sequence
  AC domain (from residue 360 to residue 493). Cytoplasmic expression of his-tagged (composed of GGHHHHHH sequence) domain.
MDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTGGHHHHHH

SEQ ID NO: 23 - protein, 698 aa, Artificial Sequence
  Adenylate cyclase fragment without His tag
MTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDP

(19) United States
(12) Patent Application Publication
(10) Pub. No.: US 2021/0220462 A1
(43) Pub. Date: Jul. 22, 2021

(54) IMMUNOGENIC PROTEINS AND COMPOSITIONS

(71) Applicant: GLAXOSMITHKLINE BIOLOGICALS SA, Rixensart (BE)

(72) Inventors: Cindy CASTADO, Rixensart (BE); Nadia OUAKED, Rixensart (BE); Steven Clement SIJMONS, Rixensart (BE)

(73) Assignee: GLAXOSMITHKLINE BIOLOGICALS SA, Rixensart (BE)

(21) Appl. No.: 17/270,328

(22) PCT Filed: Aug. 22, 2019
(86) PCT No.: PCT/EP2019/072487

§ 371 Date: Feb. 22, 2021

(30) Foreign Application Priority Data
Aug. 23, 2018 (EP) 18190597.7
Aug. 24, 2018 (EP) 18190822.9

(51) Int. Cl. A61K 39/02; A61K 39/39; A61P 31/04; C12N 9/88

(52) U.S. Cl. CPC A61K 39/099; A61K 39/39; A61K 2039/55505; C12N 9/88; C12Y 406/01001; A61P 31/04

(57) ABSTRACT

The invention provides proteins and compositions for the treatment and prevention of disease caused by Bordetella pertussis.

### TECHNICAL FIELD

The invention provides proteins and compositions for the treatment and prevention of disease caused by Bordetella pertussis.

### BACKGROUND ART

In recent years, resurgence in disease caused by Bordetella pertussis has been observed even in countries with high vaccine coverage. Whilst the precise reasons for this recurrence are not clear, potential causes include waning immunity and epidemiological changes in the circulating strains.

Adenylate cyclase is a key virulence factor of B. pertussis that disrupts normal cellular function and is critical for colonization. Adenylate cyclase is broadly conserved between different strains of B. pertussis, indeed only a single nucleotide polymorphism has been observed across clinical strains isolated between 1920 and 2010 (Bart et al 2014). In addition, antibodies to Adenylate cyclase have been found in serum samples of patients recovering from infection by Bordetella pertussis and Bordetella parapertussis. Even though it is unclear whether the presence of anti-adenylate cyclase antibodies in vaccinated children is attributable to vaccination or to previous unrecognized Bordetella infections (Farfel et al 1989, Arciniega et al 1991), patients in whom the whole cell and acellular pertussis vaccine failed had minimal adenylate cyclase antibody responses (Cherry et al 2004).

In mice, passive immunization with anti-adenylate cyclase antibodies protected mice against a lethal respiratory challenge with B. pertussis or B. parapertussis to levels similar to those seen following vaccination using a whole-cell pertussis vaccine (Guiso et al 1989). In addition, active forms of recombinant Adenylate cyclase from E. Coli were protective against B. pertussis infection of the mouse lung (Cheung et al 2006, Mac Donald-Fyall et al 2004).

Whilst Adenylate cyclase has potential as an antigen, it is not currently a component of acellular pertussis vaccines and, in addition Adenylate cyclase is a hemolysin with enzymatic activity known to impair host immune cell function.

There therefore remains a need for improved vaccines against infection with Bordetella and it is an object of the invention to provide proteins and immunogenic compositions which can be used in the development of such vaccines.

### SUMMARY OF THE INVENTION

The present invention generally relates to novel fragments of Bordetella sp. Adenylate cyclase (CyaA or ACT). The novel fragments comprise or consist of amino acid sequences having sequence identity to SEQ ID NOs: 2, 3, 4, 5, 6, 7, 15, 16, 17, 18, 19, 20, 21, 22 or 23.

In a first aspect of the invention there is provided a polypeptide that comprises or consists of an amino acid sequence:

A-X-B

wherein: X is an amino acid sequence consisting of a sequence having identity with SEQ ID NO: 2, 3, 4, 15, 16 or 17; A is an optional N terminal amino acid sequence; B is an optional C terminal amino acid sequence. Particularly, the level of sequence identity is from 90% to 100%. More particularly, the level of sequence identity is at least 90%, at least 91%, at least 92%, at least 93%, at least 94%, at least 95%, at least 96%, at least 97%, at least 98% or at least 99%. Yet more particularly, the level of sequence identity with SEQ ID NO: 2, 3 or 4 is 100%. Particularly A and B are optional sequences not derived from Adenylate cyclase, particularly the Adenylate cyclase of SEQ ID NO:1 or fragments of three or more contiguous amino acids thereof, for example, 4, 5, 6, 7, 8, 9, 10 contiguous amino acids. As used herein reference to “not derived” in the context of the invention means that the optional sequences A and/or B do not correspond with, originate from or otherwise share significant sequence identity, for example less than 50%, less than 45%, less than 40%, less than 35%, or less than 30% sequence identity with the naturally occurring sequence of adenylate cyclase provided as SEQ ID NO:1.

In some embodiments A and B are absent. In such embodiments, the polypeptide of the first aspect may consist of a sequence having at least 95%, at least 96%, at least 97%, at least 98%, at least 99% identity or 100% identity with SEQ ID NO: 2, 3, 4, 15, 16 or 17.

In some embodiments at least one of A or B is present, for example, A alone, B alone or both A and B present. In some embodiments A and/or B is a histidine tag, for example, A and/or B is Hisₙ where n=3, 4, 5, 6, 7, 8, 9, 10 or more. Thus, in some embodiments, the polypeptide consists of a sequence having at least 90%, at least 91%, at least 92%, at least 93%, at least 94%, at least 95%, at least 96%, at least 97%, at least 98%, at least 99% or 100% identity with SEQ ID NO: 5, 6, 7, 8, 21, 22 or 23. Particularly the polypeptide consists of a sequence having at least 90%, at least 91%, at least 92%, at least 93%, at least 94%, at least 95%, at least 96%, at least 97%, at least 98%, at least 99% or 100% identity with SEQ ID NO: 23.

Particularly, following immunisation, the polypeptide of the first aspect is capable of eliciting an antibody response comprising antibodies that bind to the Adenylate cyclase protein having amino acid sequence of SEQ ID NO:1.

In a second aspect of the invention, there is provided a nucleic acid encoding a polypeptide according to the first aspect.

In a third aspect of the invention, there is provided a bacterium that comprises a nucleic acid according to the second aspect. More particularly, there is provided a bacterium that comprises a nucleic acid according to the second aspect and which expresses or is capable of expressing a polypeptide according to the first aspect.

In a fourth aspect of the invention, there is provided an immunogenic composition comprising a polypeptide according to the first aspect or a nucleic acid according to the second aspect. Particularly the immunogenic composition comprises an adjuvant. Yet more particularly the immunogenic composition comprises a divalent metal salt. Still yet more particularly, the immunogenic composition will comprise a divalent metal salt wherein the divalent metal salt is a Calcium salt, for example, Calcium chloride.

In a fifth aspect of the invention, there is provided a polypeptide according to the first aspect, a nucleic acid according to the second aspect or an immunogenic composition according to the fourth aspect for use in therapy. Particularly, the polypeptide according to the first aspect, a nucleic acid according to the second aspect or an immunogenic composition according to the fourth aspect for use in treating or preventing disease and/or infection caused by Bordetella, for example, Bordetella pertussis.

In a sixth aspect of the invention, there is provided a method or treating or prevent disease and/or infection caused by Bordetella pertussis in a mammal comprising administering an effective amount of the polypeptide according to the first aspect, a nucleic acid according to the second aspect or an immunogenic composition according to the fourth aspect.

### BRIEF DESCRIPTION OF THE DRAWINGS

FIG. 1: Structure of B. pertussis Adenylate cyclase. Two lysine residues highlighted can be palmitoylated by the co-expressed protein acyl transferase CyaC. Fragment (1): AC domain of adenylate cyclase, from residue 1 to residue 400 (SEQ ID NO: 15); Fragment (2): AC domain of adenylate cyclase, from residue 1 to residue 400 indicating a GS insertion between residues 188 and 189 (SEQ ID NO: 16); Fragment (3): AC domain of adenylate cyclase, from residue 360 to residue 493 (SEQ ID NO:17); Fragment (4): RTX fragment of adenylate cyclase, from residue 985 to residue 1681 (SEQ ID NO:2)

FIG. 2: Polypeptide fragments are well adsorbed onto Al(OH)₃ at pH 7,4. Key: 1, 2—Fragment alone (NC, C); 3,4—Fragment in Al(OH)3 (NC, C); 5,6—Fragment alone treated like for formulation with AL(OH)3 (NC,C); NC—non centrifuged; C—centrifuged (supernatant). Absence of Fragment in supernatant of formulation with Al(OH)3 (Rows 3 and 4) demonstrates that the fragment is well adsorbed. Rows 5 and 6 demonstrate no visible degradation of the polypeptide fragments in conditions of formulation with Al(OH)3.

FIG. 3: Polypeptide fragment folded/unfolded conformation wo/w EGTA is detected even in presence of AS001. (1) Typical peak of fragment alone is observed (Tm˜70° C.); (2) In presence of AS01 or AS01 buffer fragment peak is preserved (a bit lower Tm)→AS01 buffer less optimal for fragment, but secondary structure is preserved (folded state); (3) In presence of EGTA loss of fragment peak and typical Tm is observed even in presence of AS01—secondary structure not preserved; (4) When formulated with Al(OH)₃, loss of fragment peak is observed—polypeptide fragment adsorbed at the surface.

FIG. 4: Results of purification of polypeptide fragments (Example 1).

FIG. 5: Results of Adenylate cyclase Toxin cytotoxicity sero-neutralization assay (Example 3).

FIG. 6: Provides a schematic of the immunisation schedule described in Example 4.

FIG. 7: Individual serum antibody titers (anti-ACT IgG) measured by ELISA at 7PII (day 28) after immunization with either full-length adenylate cyclase or the RTX Fragment.

FIG. 8: Individual serum antibody titers (anti-ACT IgG) measured by ELISA at 7PII (day 28).

FIG. 9(a) and (b): Protective efficacy against B. pertussis intranasal challenge induced by the different vaccines. As shown in FIG. 9(a), the amount of PRN is high enough to induce full protection. All the investigated formulations as well as Infanrix reduced the number of CFU with respect to the unvaccinated group. As shown in FIG. 9(b), all the investigated formulations as well as the Infanrix 1/4th HD group (positive control) reduced the number of CFU with respect to the unvaccinated group. High significant differences were observed between Infanrix 1/4th HD group (positive control) and DTPa 1/80HD with/without fragment RTX groups (with GMRs greater than 500).

FIG. 10: Expression of SEQ ID NO: 21 under the following conditions: E. Coli strain: B834(DE3), IPTG concentration: 1 mM, Induction: 16° C., Overnight. (1) Molecular Weight Marker; (2) Non-induced; (3) Induced.

FIG. 11: Purification of SEQ ID NO: 21. (1) Molecular Weight Marker; (2) 5 μg Protein; (3) 2 μg protein; (4) 1 μg protein; (6) E. coli lysate 1 μl.

FIG. 12: Expression of SEQ ID NO: 22 under the following conditions: Expression conditions: E. Coli strain: B834(DE3), IPTG concentration: 1 mM, Induction: 37° C., 3 h. (1) Molecular Weight Marker; (2) Non-induced; (3) Induced; (4) Non-induced; (5) Induced.

FIG. 13: Purification of SEQ ID NO: 22. (1) Molecular Weight Marker; (2) 5 μg protein; (3) 2 μg protein; (4) 1 μg protein; (5) E. coli lysate 1 μl.

### DETAILED DESCRIPTION

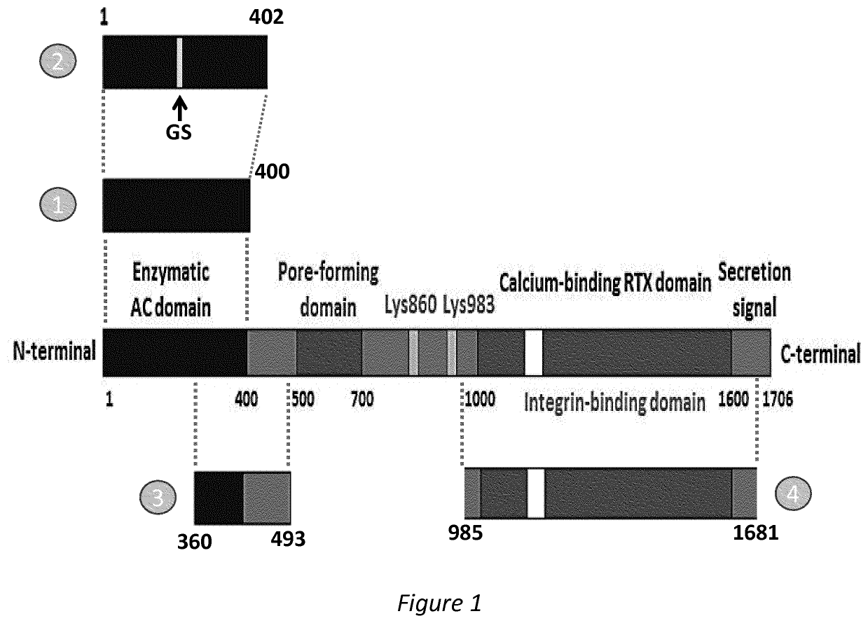

The invention relates inter alia to fragments of Adenylate cyclase for use as antigens. Adenylate cyclase is a multifunctional protein with a length of 1706 amino acids (Sebo et al, 2014). It consists of a N-terminal enzymatic adenylate cyclase (AC) domain (residues 1-400), a hydrophobic pore-forming domain (residues 500-700), a fatty acyl-modified domain (residues 800-1000), a calcium-binding repeat-in-toxin (RTX) domain (residues 1000-1600) and a C-terminal, uncleaved secretion signal (FIG. 1). The C-terminal 1300 residues are also referred to as the hemolysin (Hly) moiety. The Hly moiety binds to the Complement Receptor 3 on the host cell through an integrin-binding region in the RTX domain and enables translocation of the AC domain into the host cell cytosol, resulting in the unregulated conversion of ATP to cAMP. In addition, the pore-forming domain can oligomerize and form small, cation-selective pores in the host cell membranes, resulting in moderate hemolysis.

The inventors have now succeeded in identifying fragments of the full-length Adenylate cyclase (SEQ ID NO: 1) that retain immunogenicity whilst avoiding toxicity, such as 5 hemolysis, associated with the full-length protein.

Fragments of Adenylate cyclase that contain epitopes responsible for protection are provided as SEQ ID NOs: 2, 3, 4, 15, 16, 17, 18, 19, 20, 21, 22 or 23 herein.

The amino acid sequence of SEQ ID NO:2 is a 696 amino acid fragment equating to amino acids from residue 985 to residue 1681 of the wild-type Adenylate cyclase sequence given in SEQ ID NO:1. SEQ ID NOs: 3 and 4 comprise one or two additional Glycine residues respectively. SEQ ID NO:23 includes an N-terminal methionine residue.

The amino acid sequence of SEQ ID NO:15 is an amino acid fragment equating to amino acids from residue 1 to residue 400 of the wild-type Adenylate cyclase sequence given in SEQ ID NO:1 (the Methionine residue corresponding with position 1 of SEQ ID NO: 1 is not shown in SEQ ID NO:15 but in some embodiments of the invention may be included as N terminal sequence ‘A’).

The amino acid sequence of SEQ ID NO:16 is an amino acid fragment equating to amino acids from residue 1 to residue 400 of the wild-type Adenylate cyclase sequence given in SEQ ID NO:1 further comprising a GS insertion between residues 188 and 189 of SEQ ID NO:1 (the Methionine residue corresponding with position 1 of SEQ ID NO: 1 is not shown in SEQ ID NO:16 but in some embodiments of the invention may be included as N terminal sequence ‘A’).

The amino acid sequence of SEQ ID NO:17 is an amino acid fragment equating to amino acids from residue 360 to residue 493 of the wild-type Adenylate cyclase sequence given in SEQ ID NO:1 (in some embodiments of the invention a methionine residue may be included as N terminal sequence ‘A’).

According to the invention, therefore, a polypeptide is provided comprising an amino acid sequence:

A-X-B

wherein: X is an amino acid sequence consisting of a sequence having at least 90% identity with SEQ ID NO: 2, 3, 4, 15, 16 or 17; A is an optional N terminal amino acid sequence; B is an optional C terminal amino acid sequence, and wherein A and B are not derived from adenylate cyclase or a fragment thereof.

In embodiments relating to RTX fragments, particularly X is an amino acid sequence that has no more than 698 contiguous amino acids from SEQ ID NO:1. More particularly, X is an amino acid sequence that has from 691 to 698 contiguous amino acids from SEQ ID NO:1. Yet more particularly, X is an amino acid sequence that has at least 691 to no more than 698 contiguous amino acids from SEQ ID NO:1. Still yet more particularly, such RTX fragments have at least 90% identity, at least 91%, at least 92%, at least 93%, at least 94%, at least 95%, at least 96%, at least 97%, at least 98%, at least 99% or 100% identity to SEQ ID NO:2, 3 or 4.

In embodiments relating to AC Domain fragments, particularly X is an amino acid sequence that has no more than 400 contiguous amino acids from SEQ ID NO:1. More particularly, X is an amino acid sequence that has less than 400 contiguous amino acids from SEQ ID NO:1. Still yet more particularly, X at least 90% identity, at least 91%, at least 92%, at least 93%, at least 94%, at least 95%, at least 96%, at least 97%, at least 98%, at least 99% or 100% identity to SEQ ID NO:15, 16 or 17.

Sequence identity may be determined using a pairwise alignment algorithm, each moving window of x amino acids from N-terminus to C-terminus (such that for an alignment that extends to p amino acids, where p>x, there are p-x+1 such windows) has at least x⋅y identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x⋅y is not an integer then it is rounded up to the nearest integer. The preferred pairwise alignment algorithm is the

Needleman-Wunsch global alignment algorithm [1], using default parameters (e.g. with Gap opening penalty=10.0, and with Gap extension penalty=0.5, using the EBLOSUM62 scoring matrix). This algorithm is conveniently implemented in the needle tool in the EMBOSS package [2]. With regard to sequences of the invention, these being fragments of adenylate cyclase, sequence identity should be calculated with respect to and along the entire (i.e. full) length of the longer sequence, for example the full-length or wild-type sequence.

For the avoidance of doubt, amino acid sequences of full length, native, Adenylate cyclase of Bordetella pertussis, for example SEQ ID NO: 1, are specifically excluded from the scope of the invention.

The amino acid sequence of -A- or -B- will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. Useful linkers are GSGS (SEQ ID NO: 9), GSGGGG (SEQ ID NO: 10) or GSGSGGGG (SEQ ID NO: 11), with the Gly-Ser dipeptide being formed from a BamHI restriction site, thus aiding cloning and manipulation, and the (Gly)₄ tetrapeptide being a typical poly-glycine linker. Other suitable linkers include a Leu-Glu dipeptide or Gly-Ser. Linkers may contain at least one glycine residue to facilitate structural flexibility e.g. 1, 2, 3, 4, 5, 6, 7, 8, 9, 10 or more glycine residues. Such glycines may be arranged to include at least two consecutive glycines in a Gly-Gly dipeptide sequence, or a longer oligo-Gly sequence i.e. Glyₙ where n=2, 3, 4, 5, 6, 7, 8, 9, 10 or more.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 20 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). In some embodiments, -A- is a heterologous signal peptide coming from NspA (an outer membrane protein of Neisseria meningitidis). The signal peptide may include one or two additional amino acids coming from NspA to optimise signal peptide cleavage. Other suitable N-terminal amino acid sequences will be apparent to those skilled in the art. If X lacks its own N-terminus methionine, -A- is preferably an oligopeptide (e.g. with 1, 2, 3, 4, 5, 6, 7 or 8 amino acids) which provides a N-terminus methionine e.g. Met-Ala-Ser, or a single Met residue. In a nascent polypeptide the -A- moiety can provide the polypeptide's N-terminal methionine (formyl-methionine, fMet, in bacteria). For example, when X is SEQ ID NOs: 2, 3, 4, 5, 6, 7, 15, 16 or 17, in certain embodiments it is envisaged that -A- may provide or be such an N-terminal methionine (for example, as SEQ ID NOs: 18, 19, 20, 21 or 22). One or more amino acids may be cleaved from the N-terminus of a nascent -A- moiety, however, such that the -A- moiety in a mature polypeptide of the invention does not necessarily include a N-terminal methionine.

-B- is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Particular His tags suitable for use in the invention include GGHHHHHH (SEQ ID NO: 12), GHHHHHH (SEQ ID NO: 13), HHHHHH (SEQ ID NO: 14) and the like. Other suitable C-terminal amino acid sequences will be apparent to those skilled in the art, such as a glutathione-S-transferase, thioredoxin, 14kDa fragment of S. aureus protein A, a biotinylated peptide, a maltose-binding protein, an enterokinase flag, etc.

Polypeptide fragments of the invention including -A- and/or -B- include SEQ ID NOs: 5, 6, 7, 8, 18, 19, 20, 21, 22 or 23. Suitable fragments or polypeptides including -A- and/or -B- may consist of a polypeptide having at least 90% sequence identity, at least 91%, at least 92%, at least 93%, at least 94%, at least 95%, at least 96%, at least 97%, at least 98%, at least 99% or 100% sequence identity to SEQ ID NO:5, 6, 7, 8, 18, 19, 20, 20 21, 22 or 23.

As discussed above, the polypeptides of the invention may comprise additional polypeptide sequences at the -N and/or -C terminus which are not derived from Adenylate cyclase of SEQ ID NO:1. Thus, reference to such polypeptide sequences not derived from Adenylate cyclase may be understood to mean that the additional polypeptide sequence does not comprise a contiguous sequence of three or more, for example, four, five, six, seven, eight, nine or ten contiguous amino acids of SEQ ID NO:1.

An “epitope” is the part of a polypeptide that is recognised by the immune system and that elicits an immune response. Thus, the polypeptides of the invention comprise epitopes that will, when administered to a subject, elicit an antibody response comprising antibodies that bind to the wild-type Adenylate cyclase protein having amino acid sequence SEQ ID NO: 1. The polypeptides of the invention are thus capable of competing with both SEQ ID NO: 1 for binding to an antibody raised against SEQ ID NO: 1.

Antibodies can readily be generated against the polypeptides of the invention using standard immunisation methods and the ability of these antibodies to bind to the wild-type Adenylate cyclase protein of SEQ ID NO: 1 can be assessed using standard assays such as ELISA assays. Similarly, the ability of polypeptides to compete with antibodies raised against the wild-type Adenylate cyclase protein can be readily determined using competition assay techniques known in the art, including equilibrium methods such as ELISA, kinetic methods such as BIACORE® and by flow cytometry methods. A polypeptide that competes with wild-type Adenylate cyclase protein of SEQ ID NO: 1 for binding to an antibody will cause a reduction in the observed total binding of the wild-type protein to the antibody, compared to when the polypeptide is not present. Typically, this reduction in binding is 10% or greater, 20% or greater, 30% or greater, 40% or greater, 60% or greater, for example a reduction in binding of 70% or more in the presence of the polypeptide of the invention compared to antibody binding observed for the protein having SEQ ID NO:1. The ability of the polypeptides of the invention to induce protection against strains of Bordetella pertussis can also be confirmed in animal models known in the art.

The polypeptides of the invention may, compared with SEQ ID NO: 1 include at least one, for example, one, two, three, four, five, six or seven conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity.

The polypeptides of the invention may have at least one, for example, one, two, three, four, five, six or seven single amino acid deletions relative to fragments of SEQ ID NO: 1.

The polypeptides may also include at least one, for example, one, two, three, four, five, six or seven insertions relative to equivalent sequences of SEQ ID NO: 1. For example, certain embodiments relating to fragments of the AC domain of Adenylate cyclase will comprise a modification to knock-out or reduce the function or activity of this domain, for example, calmodulin activity (Ladant D, Glaser P, Ullmann A. J. Biol. Chem. 1992, 267:2244-50). Particular examples of fragments and polypeptides of the invention comprising an insertion include SEQ ID NOs: 16, 19 and 21. These sequences comprise a GS (Glycine-Serine) insertion between residues 188 and 189 relative to SEQ ID NO: 1. Activity or function of the AC domain may be determined by assays known in the art, for example, as disclosed in Fiser et al. J Biol Chem. 2007 Feb 2;282(5):2808-20.

The polypeptides of the invention may be prepared in many ways known to the skilled person, for example, by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. Particularly, biological synthesis may be used e.g. the polypeptides may be produced by translation. This may be carried out in vitro or in vivo. Polypeptides may have covalent modifications at the C-terminus and/or N-terminus. Polypeptides can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides are preferably provided in purified or substantially purified form i.e. substantially free from other polypeptides (e.g. free from naturally-occurring polypeptides), particularly from other Bordetella or host cell polypeptides, and are generally at least about 50% pure (by weight), and usually at least about 90% pure, for example, at least about 91% pure (by weight), at least about 92% pure (by weight), at least about 93% pure (by weight), at least about 94% pure (by weight), at least about 95% pure (by weight), at least about 96% pure (by weight), at least about 97% pure (by weight), at least about 98% pure (by weight), at least about 99% pure (by weight), at least about 99.5% pure (by weight), at least about 99.9% pure (by weight) i.e. less than about 50%, and more preferably less than about 10% (e.g. 5% or less) of a composition is made up of other expressed polypeptides.

Polypeptides may be attached to a solid support. Polypeptides may comprise a detectable label (e.g. a radioactive or fluorescent label, or a biotin label). Polypeptides can be naturally or non-naturally glycosylated (i.e. the polypeptide has a glycosylation pattern that differs from the glycosylation pattern found in the corresponding naturally occurring polypeptide).

The invention also provides a process for producing polypeptides of the invention, comprising culturing a bacterium of the invention under conditions which induce polypeptide expression. Although expression of the polypeptide may take place in a Bordetella bacterium, the invention may use a heterologous host for expression. The heterologous host may be prokaryotic (e.g. a bacterium) or eukaryotic. It will usually be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

The invention also provides a process for producing a polypeptide of the invention, wherein the polypeptide is synthesised in part or in whole using chemical means.

The invention also provides a composition comprising at least one polypeptide of the invention.

### Nucleic Acids

The invention also provides a nucleic acid comprising a nucleotide sequence encoding a polypeptide or a hybrid polypeptide of the invention.

For example, the invention provides a nucleic acid comprising a nucleotide sequence encoding a polypeptide comprising or consisting of an amino acid sequence selected from the group consisting of: SEQ ID NO:2, SEQ ID NO:3, SEQ ID NO:4, SEQ ID NO:5, SEQ ID NO:6, SEQ ID NO:7, SEQ ID NO:15, SEQ ID NO:16, SEQ ID NO:17, SEQ ID NO:18, SEQ ID NO:19, SEQ ID NO:20, SEQ ID NO:21, SEQ ID NO:22 and

SEQ ID NO:23. For the avoidance of doubt, nucleic acid sequences encoding full length Adenylate cyclase, for example SEQ ID NO: 1, are not part of the invention and are excluded. The invention also provides nucleic acids comprising nucleotide sequences having sequence identity to such nucleotide sequences. Such nucleic acids include those using alternative codons to encode the same amino acid. In particular, nucleic acids may contain alternative codons optimised for expression in specific microorganisms, e.g. E. coli.

The invention also provides nucleic acid which can hybridize to these nucleic acids. Hybridization reactions can be performed under conditions of different “stringency”. Conditions that increase stringency of a hybridization reaction of widely known and published in the art. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see refs 3 & 32, etc.].

The invention includes nucleic acid comprising sequences complementary to these sequences (e.g. for antisense or probing, or for use as primers).

Nucleic acids according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other Bordetella or host cell nucleic acids, generally being at least about 50% pure (by weight), and usually at least about 90% pure.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids.

A “host cell” includes an individual cell which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Nucleic acids of the invention can be used, for example: to produce polypeptides in vitro or in vivo; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides vectors comprising nucleotide sequences of the invention (e.g. cloning or expression vectors) and bacteria and other host cells transformed with such vectors.

### Immunogenic Compositions

The polypeptides of the invention are useful as active ingredients in immunogenic compositions. The term “immunogenic composition” broadly refers to any composition that may be administered to elicit an immune response, such as an antibody or cellular immune response, against an antigen present in the composition. Thus, compositions of the invention are immunogenic. When the immunogenic compositions prevent, ameliorate, palliate or eliminate disease from the subject, then such compositions may be referred to as a vaccine. Vaccines according to the invention may either be prophylactic (i.e. to prevent infection) or therapeutic (i.e. to treat infection), but will typically be prophylactic. The term “prevent infection” as used in the context of the present invention, means that the immune system of a subject has been primed (e.g. by vaccination) to trigger an immune response and repel the infection. Thus, it will be clear to those skilled in the art that a vaccinated subject may thus get infected, but is better able to repel the infection than a control subject. In certain embodiments, the immunogenic composition is a vaccine. The term “antigen” refers to a substance that, when administered to a subject, elicits an immune response directed against the substance. In the context of the present invention, polypeptides of the invention are antigens. Preferably the antigens are recombinant antigens prepared or manufactured using recombinant DNA technology. Particularly, when administered to a subject the immunogenic composition elicits an immune response directed against Bordetella and more particularly against a fragment of SEQ ID NO: 1. Particularly the immune response directed against Bordetella is protective, that is, it can prevent or reduce infection, disease or colonisation caused by Bordetella, particularly Bordetella pertussis. Compositions may thus be pharmaceutically acceptable. They will usually include components in addition to the antigens e.g. they typically include one or more pharmaceutical carrier(s) and/or excipient(s). Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus, a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To control tonicity, it is preferred to include a physiological salt, such as a sodium salt. Sodium chloride (NaCl) may be used, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Immunogenic compositions of the invention may comprise a divalent metal salt, more particularly a Calcium salt, yet more particularly Calcium chloride. The presence of Ca²⁺ might useful for maintaining conformation of the polypeptide fragments of the invention.

Compositions will generally have an osmolality of between 200 mOsm/kg and 400 mOsm/kg, preferably between 240-360 mOsm/kg, and will more preferably fall within the range of 290-310 mOsm/kg. In some embodiments, the compositions may be hypertonic, for example having an osmolarity of around 700 mOsm/Kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below.

Adjuvants which may be used in compositions of the invention include, mineral containing compositions such as aluminium salts and calcium salts. The compositions of the invention may include mineral salts such as hydroxides (e.g. oxyhydroxides), phosphates (e.g. hydroxyphosphates, orthophosphates), sulphates, etc. [e.g. see chapters 8 & 9 of ref. 4], or mixtures of different mineral compounds, with the compounds taking any suitable form (e.g. gel, crystalline, amorphous, etc.). The mineral containing compositions may also be formulated as a particle of metal salt.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ [chapter 9 of ref. 4]. The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls [ch. 9 of ref. 4].

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

In one embodiment, an adjuvant component includes a mixture of both an aluminium hydroxide and an aluminium phosphate. In this case there may be more aluminium phosphate than hydroxide e.g. a weight ratio of at least 2:1 e.g. ≥5:1, ≥6:1, ≥7:1, ≥8:1, ≥9:1, etc.

The concentration of Al⁺⁺⁺ in a composition for administration to a patient is preferably less than 10 mg/ml e.g. ≤5 mg/ml, ≤4 mg/ml, ≤3 mg/ml, ≤2 mg/ml, ≤1 mg/ml, etc. A preferred range is between 0.3 and 1 mg/ml. A maximum of <0.85 mg/dose is preferred.

Polypeptides of the invention may be adsorbed to an aluminium adjuvant, such as Al(OH)₃.

Oil emulsion compositions suitable for use as adjuvants in the invention include squalene-water emulsions, such as MF59 [Chapter 10 of ref. 4; see also ref. 5] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

AS01 is an Adjuvant System containing MPL (3-O-desacyl-4′- monophosphoryl lipid A), QS21 ((Quillaja saponaria Molina, fraction 21) Antigenics, New York, N.Y., USA) and liposomes. AS01B is an Adjuvant System containing MPL, QS21 and liposomes (50 μg MPL and 50 μg QS21). AS01E is an Adjuvant System containing MPL, QS21 and liposomes (25 μg MPL and 25 μg QS21). In one embodiment, the immunogenic composition or vaccine comprises AS01. In another embodiment, the immunogenic composition or vaccine comprises AS01B or AS01E. In a particular embodiment, the immunogenic composition or vaccine comprises AS01E.

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 6. Saponin formulations may also comprise a sterol, such as cholesterol [7].

Combinations of saponins and cholesterols can be used to form unique particles called immunostimulating complexs (ISCOMs) [chapter 23 of ref. 4]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 7-9. Optionally, the ISCOMS may be devoid of additional detergent [10].

A review of the development of saponin based adjuvants can be found in refs. 11 & 12.

Other adjuvants suitable for use in the invention include bacterial or microbial derivatives such as non-toxic derivatives of enterobacterial lipopolysaccharide (LPS),

Lipid A derivatives, immunostimulatory oligonucleotides, ADP-ribosylating toxins and detoxified derivatives thereof and bacterial Outer Membrane Vesicles (OMV).

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 13. Such “small particles” of 3dMPL are small enough to be sterile filtered through a 0.22 μm membrane [13]. Other non-toxic LPS derivatives include monophosphoryl lipid A mimics, such as aminoalkyl glucosaminide phosphate derivatives e.g. RC-529 [14,15].

Lipid A derivatives include derivatives of lipid A from Escherichia coli such as OM-174. OM-174 is described for example in refs. 16 & 17. Examples of liposome formulations suitable for use as adjuvants are described in refs. 18-20.

In certain embodiments, the antigens and adjuvants in a composition will be in admixture at the time of delivery to a patient. In certain embodiments, the adjuvant and antigen may be kept separately in a packaged or distributed vaccine, ready for final formulation at the time of use. The antigen may be in an aqueous or lyophilised form, such that the vaccine is finally prepared by mixing two components prior to administration. The volume ratio of the two liquids for mixing can vary (e.g. between 5:1 and 1:5) but is generally about 1:1.

The invention may also comprise combinations of aspects of one or more of the adjuvants identified above. For example, the following adjuvant compositions may be used in the invention: (1) a saponin and an oil-in-water emulsion [21]; (2) a saponin (e.g. QS21)+a non-toxic LPS derivative (e.g. 3dMPL) [22]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [23]; (5) combinations of 3dMPL with, for example, QS21 and/or oil-in-water emulsions [24];

Compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for pulmonary administration e.g. as an inhaler, using a fine powder or a spray. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Methods of Treatment, and Administration of the Vaccine

The invention also provides a method for raising an immune response in a mammal comprising the step of administering an effective amount of a polypeptide, nucleic acid or an immunogenic composition as described above. The immune response is preferably protective and preferably involves antibodies and/or cell-mediated immunity. The method may raise a booster response.

The invention also provides a polypeptide, nucleic acid or an immunogenic composition described above for use as a medicament e.g. for use in raising an immune response in a mammal.

The invention also provides the use of a polypeptide, nucleic acid or an immunogenic composition described above in the manufacture of a medicament for raising an immune response in a mammal.

By raising an immune response in the mammal by these uses and methods, the mammal can be protected against disease and/or infection caused by Bordetella, particularly Bordetella pertussis e.g. against whooping cough.

The invention also provides a delivery device pre-filled with an immunogenic composition of the invention.

The mammal is preferably a human. The human may be a child, teenager or an adult. The child may be less than one year old, for example, a new born from 0 to 60 days old, from 0 to 8 weeks, for example 7 weeks old, from two to eighteen months of age, for example, two, three, four, five, six, fifteen, sixteen, seventeen, eighteen months of age. Immunogenic compositions of the invention may be for use as a booster vaccine, for example, administered at four to six years of age (US schedule). In the UK, pertussis vaccinations are given at 2, 3, and 4 months, with a pre-school booster at 3 years 4 months.

Compositions of the invention will generally be administered directly to a patient. Direct delivery may be accomplished by parenteral injection (e.g. subcutaneously, intraperitoneally, intravenously, intramuscularly, or to the interstitial space of a tissue). In some embodiments, mucosal administration may be used.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, less than 5 years old, 1-5 years old, 5-15 years old, 15-55 years old, or at least 55 years old. Preferred patients for receiving the vaccines are adolescents (e.g. 13-20 years old), pregnant women, and the elderly (e.g. ≥50 years old, ≥60 years old, and preferably ≥65 years. The vaccines are not suitable solely for these groups, however, and may be used more generally in a population.

Combinations with Other Antigens

The polypeptides of the invention may be used in combination with other antigens. Thus, the invention provides an immunogenic composition comprising a combination of:
(1) a polypeptide of the invention as discussed above; and
(2) one or more antigen(s) selected from the group consisting of: diphtheria toxoid;

tetanus toxoid; one or more pertussis antigens; hepatitis B virus surface antigen; an inactivated poliovirus antigen; a conjugate of the capsular saccharide antigen from serogroup B of Haemophilus influenzae; one or more RSV antigens.

Diphtheria toxoid can be obtained by treating (e.g. using formaldehyde) diphtheria toxin from Corynebacterium diphtheriae. Diphtheria toxoids are disclosed in more detail in, for example, chapter 13 of reference 25.

Tetanus toxoid can be obtained by treating (e.g. using formaldehyde) tetanus toxin from Clostridium tetani. Tetanus toxoids are disclosed in more detail in chapter 27 of reference 25.

Pertussis antigens in vaccines are either cellular (whole cell, Pw) or acellular (Pa). The invention can use either sort of pertussis antigen. Preparation of cellular pertussis antigens is well documented (e.g. see chapter 21 of reference 25) e.g. it may be obtained by heat inactivation of phase I culture of B. pertussis. Acellular pertussis antigen(s) comprise specific purified B. pertussis antigens, either purified from the native bacterium or purified after expression in a recombinant host. It is usual to use more than one acellular antigen, and so a composition may include one, two or three of the following well-known and well-characterized B. pertussis antigens: (1) detoxified pertussis toxin (pertussis toxoid, or ‘PT’), including genetically detoxified pertussis toxoid; (2) filamentous hemagglutinin (FHA); (3) pertactin (also known as the ‘69 kiloDalton outer membrane protein’). FHA and pertactin may be treated with formaldehyde prior to use according to the invention. PT may be detoxified by treatment with formaldehyde and/or glutaraldehyde but, as an alternative to this chemical detoxification procedure, it may be a mutant PT in which enzymatic activity has been reduced by mutagenesis [26]. Further acellular pertussis antigens that can be used include fimbriae (e.g. agglutinogens 2 and 3).

Hepatitis B virus surface antigen (HBsAg) is the major component of the capsid of hepatitis B virus. It is conveniently produced by recombinant expression in a yeast, such as a Saccharomyces cerevisiae.

Inactivated poliovirus (IPV) antigens are prepared from viruses grown on cell culture and then inactivated (e.g. using formaldehyde). Because poliomyelitis can be caused by one of three types of poliovirus, as explained in chapter 24 of reference 25, a composition may include three poliovirus antigens: poliovirus Type 1 (e.g. Mahoney strain), poliovirus Type 2 (e.g. MEF-1 strain), and poliovirus Type 3 (e.g. Saukett strain).

When a composition includes one of diphtheria toxoid, tetanus toxoid or an acellular pertussis antigen in component (2) then it will usually include all three of them i.e. component (2) will include a D-T-Pa combination.

When a composition includes one of diphtheria toxoid, tetanus toxoid or a cellular pertussis antigen in component (2) then it will usually include all three of them i.e. component (2) will include a D-T-Pw combination.

### Specific Embodiments of the Invention—AC Domain Fragments

In embodiments of the invention there is provided a polypeptide that comprises or consists of an amino acid sequence:

A-X-B

wherein: X is an amino acid sequence consisting of a sequence having identity with SEQ

ID NO: 15, 16 or 17; A is an optional N terminal amino acid sequence; B is an optional C terminal amino acid sequence. Particularly, the level of sequence identity is from 90% to 100%. More particularly, the level of sequence identity is at least 90%, at least 91%, at least 92%, at least 93%, at least 94%, at least 95%, at least 96%, at least 97%, at least 98% or at least 99%. Yet more particularly, the level of sequence identity is 100%. Particularly A and B are optional sequences not derived from Adenylate cyclase, particularly the Adenylate cyclase of SEQ ID NO:1 or fragments of three or more contiguous amino acids thereof. In some embodiments, the novel fragments comprise or consist of amino acid sequences having sequence identity to SEQ ID NOs: 15, 16, 17, 18, 19, 20. 21 or 22.

### EMBODIMENT 1

A polypeptide comprising an amino acid sequence:

A-X-B

wherein: X is an amino acid sequence consisting of a sequence having at least 99% identity with SEQ ID NO: 15, 16 or 17; A is an optional N terminal amino acid sequence; B is an optional C terminal amino acid sequence, and wherein, when present, A and B are not derived from adenylate cyclase or a fragment thereof.

### EMBODIMENT 2

The polypeptide of Embodiment 1 which consists of a sequence having at least 99% identity with SEQ ID NO: 15, 16, 17, 18, 19 or 20.

### EMBODIMENT 3

The polypeptide of Embodiment 2 which consists of a sequence having 100% identity with SEQ ID NO: 15, 16, 17, 18, 19 or 20.

### EMBODIMENT 4

The polypeptide of Embodiment 1 wherein A and/or B is a histidine tag.

### EMBODIMENT 5

The polypeptide of Embodiment 4 which consists of a sequence having at least 99% identity with SEQ ID NO: 21 or 22.

### EMBODIMENT 6

The polypeptide of Embodiment 5 which consists of a sequence having 100% identity with SEQ ID NO: 21 or 22.

### EMBODIMENT 7

The polypeptide of anyone of Embodiments 1 to 6 capable of eliciting an antibody response comprising antibodies that bind to the Adenylate cyclase protein having amino acid sequence of SEQ ID NO:1, for example, as measured by adenylate cyclase toxin neutralisation assay, particularly as described in the Examples.

### EMBODIMENT 8

A nucleic acid encoding a polypeptide according to any one of Embodiments 1 to 7.

### EMBODIMENT 9

A bacterium that comprises the nucleic acid of Embodiment 8 and/or expresses a polypeptide according to any one of Embodiments 1 to 7.

### EMBODIMENT 10

An immunogenic composition comprising a polypeptide according to any one of Embodiments 1 to 7 or a nucleic acid according to claim 8.

### EMBODIMENT 11

The immunogenic composition according to Embodiment 10 which comprises an adjuvant.

### EMBODIMENT 12

A polypeptide according to any one of Embodiments 1 to 7, a nucleic acid according to Embodiment 8 or an immunogenic composition according to Embodiments 10 to 13 for use in therapy.

### EMBODIMENT 13

A polypeptide according to any one of Embodiments 1 to 7, a nucleic acid according to Embodiment 8 or an immunogenic composition according to Embodiments 10 to 13 for use in treating or preventing disease and/or infection caused by Bordetella pertussis.

### EMBODIMENT 14

A method of treating disease and/or infection caused by Bordetella pertussis in a mammal, e.g. a human, comprising administering an effective amount of the polypeptide according to any one of Embodiments 1 to 7, a nucleic acid according to Embodiment 8 or an immunogenic composition according to Embodiments 10 to 13.

### EMBODIMENT 15

A method of preventing disease and/or infection caused by Bordetella pertussis in a mammal, e.g. a human, comprising administering an effective amount of the polypeptide according to any one of Embodiments 1 to 7, a nucleic acid according to Embodiment 8 or an immunogenic composition according to Embodiments 10 to 13.

### EMBODIMENT 16

The polypeptide according to any one of Embodiments 1 to 7, a nucleic acid according to Embodiment 8 or an immunogenic composition according to Embodiments 10 to 13 for use in a method of treating or prevent disease and/or infection caused by Bordetella pertussis in a mammal, e.g. a human comprising administering an effective amount of the polypeptide, nucleic acid or immunogenic composition.

### Specific Embodiments of the Invention—RTX Fragments

In some embodiments of the invention there is provided a polypeptide that comprises or consists of an amino acid sequence:

A-X-B

wherein: X is an amino acid sequence consisting of a sequence having identity with SEQ ID NO: 2, 3 or 4; A is an optional N terminal amino acid sequence; B is an optional C terminal amino acid sequence. Particularly, the level of sequence identity is from 90% to 100%. More particularly, the level of sequence identity is at least 90%, at least 91%, at least 92%, at least 93%, at least 94%, at least 95%, at least 96%, at least 97%, at least 98% or at least 99%. Yet more particularly, the level of sequence identity is 100%. Particularly A and B are optional sequences not derived from Adenylate cyclase, particularly the Adenylate cyclase of SEQ ID NO:1 or fragments of three or more contiguous amino acids thereof.

Particularly, fragments of the invention comprise a truncation at the C-terminus of at least 20 amino acids, for example, at least 21 amino acids, at least 22 amino acids, at least 23 amino acids, at least 24 amino acids or at least 25 amino acids when compared with SEQ ID NO: 1. Particularly, fragments of the invention comprise a truncation at the C-terminus of at least 25 amino acids when compared with SEQ ID NO: 1.

In some embodiments, the novel fragments consist of amino acid sequences having sequence identity to SEQ ID NOs: 2, 3, 4, 5, 6 or 7.

### EMBODIMENT 17.

An isolated polypeptide consisting of an amino acid sequence A-X-B wherein: A is an N terminal methionine residue, X is an amino acid sequence selected from the group consisting of SEQ ID NO: 2, 3 and 4 and wherein B is absent.

### EMBODIMENT 18.

An isolated polypeptide having at least 95% sequence identity with SEQ ID NO:23.

### EMBODIMENT 19.

An isolated polypeptide consisting of the amino acid sequence of SEQ ID NO:23.

### EMBODIMENT 20:

An isolated polypeptide consisting of an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7 and SEQ ID NO:8.

### EMBODIMENT 21

The isolated polypeptide of any one of Embodiments 17 to 20, for use in prevention or reducing infection or colonisation caused by Bordetella, particularly Bordetella pertussis.

### EMBODIMENT 22

An immunogenic composition comprising (i) the isolated polypeptide of any one of Embodiments 17 to 20, (ii) diphtheria toxoid, (iii) tetanus toxoid, (iv) detoxified pertussis toxin (pertussis toxoid, or ‘PT’), particularly genetically detoxified pertussis toxoid (PTg), (v) filamentous hemagglutinin (‘FHA’) and (vi) pertactin.

### EMBODIMENT 23

An immunogenic composition comprising (i) the isolated polypeptide of any one of Embodiments 17 to 20, (ii) diphtheria toxoid, (iii) tetanus toxoid, (iv) detoxified pertussis toxin (pertussis toxoid, or ‘PT’), particularly genetically detoxified pertussis toxoid (PTg), (v) filamentous hemagglutinin (‘FHA’), (vi) pertactin and (vii) Inactivated Polio Virus (“IPV”).

### EMBODIMENT 24

An immunogenic composition comprising (i) the isolated polypeptide of any one of Embodiments 17 to 20, (ii) diphtheria toxoid, (iii) tetanus toxoid, (iv) detoxified pertussis toxin (pertussis toxoid, or ‘PT’), particularly genetically detoxified pertussis toxoid (PTg), (v) filamentous hemagglutinin (‘FHA’), (vi) pertactin (vii) Inactivated Polio Virus (“IPV”) and (viii) a Hib conjugate.

### EMBODIMENT 25

An immunogenic composition comprising (i) the isolated polypeptide of any one of Embodiments 17 to 20, (ii) diphtheria toxoid, (iii) tetanus toxoid, (iv) detoxified pertussis toxin (pertussis toxoid, or ‘PT’), particularly genetically detoxified pertussis toxoid (PTg), (v) filamentous hemagglutinin (‘FHA’), (vi) pertactin (vii) Inactivated Polio Virus (“IPV”), (viii) a Hib conjugate and (ix) hepatitis B surface antigen.

### EMBODIMENT 26

The immunogenic composition of any one of Embodiments 22 to 25, further comprising an adjuvant, for example, AS01, an aluminium salt adjuvant and/or a TLR agonist, for example, a TLR7 agonist.

### EMBODIMENT 27

The immunogenic composition of Embodiment 26. Wherein the adjuvant is an aluminium salt adjuvant selected from the group consisting of aluminium hydroxide, aluminium phosphate and aluminium hydroxyphosphate sulfate.

### EMBODIMENT 28

The immunogenic composition of any one of Embodiments 22 to 27 wherein the isolated polypeptide consists of an amino acid sequence of SEQ ID NO: 23.

### EMBODIMENT 29

A method of treating disease and/or infection caused by Bordetella pertussis in a mammal, e.g. a human, comprising administering an effective amount of the polypeptide according to any one of Embodiments 17 to 20 or the immunogenic composition of any one of Embodiments 22 to 28.

### EMBODIMENT 30

A method of preventing disease and/or infection caused by Bordetella pertussis in a mammal, e.g. a human, comprising administering an effective amount of the polypeptide according to any one of Embodiments 17 to 20 or the immunogenic composition of any one of Embodiments 22 to 28.

### EMBODIMENT 31

The polypeptide according to any one of Embodiments 17 to 20 for use in a method of treating or prevent disease and/or infection caused by Bordetella pertussis in a mammal, e.g. a human comprising administering an effective amount of the polypeptide or the immunogenic composition of any one of Embodiments 22 to 28.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references 27-34, etc.

“GI” numbering is used above. A GI number, or “GenInfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) then it receives a new GI number. Thus, the sequence associated with a given GI number is never changed.

Where the invention concerns an “epitope”, this epitope may be a B-cell epitope and/or a T-cell epitope. Such epitopes can be identified empirically (e.g. using PEPSCAN [35,36] or similar methods), or they can be predicted (e.g. using the Jameson-Wolf antigenic index [37], matrix-based approaches [38], MAPITOPE [39], TEPITOPE [40,41], neural networks [42], OptiMer & EpiMer [43, 44], ADEPT [45], Tsites [46], hydrophilicity [47], antigenic index [48] or the methods disclosed in references 49-53, etc.). Epitopes are the parts of an antigen that are recognised by and bind to the antigen binding sites of antibodies or T-cell receptors, and they may also be referred to as “antigenic determinants”.

The term “comprising” encompasses “including” e.g. a composition “comprising” X may include something additional e.g. X+Y. The word “substantially” does not exclude “completely” e.g. a composition which is “substantially free” from Y may be completely free from Y. In some implementations, the term “comprising” refers to the inclusion of the indicated active agent, such as recited polypeptides, as well as inclusion of other active agents, and pharmaceutically acceptable carriers, excipients, emollients, stabilizers, etc., as are known in the pharmaceutical industry. In some implementations, the term “consisting essentially of” refers to a composition, whose only active ingredient is the indicated active ingredient(s), for example antigens, however, other compounds may be included which are for stabilizing, preserving, etc. the formulation, but are not involved directly in the therapeutic effect of the indicated active ingredient. Use of the transitional phrase “consisting essentially” means that the scope of a claim is to be interpreted to encompass the specified materials or steps recited in the claim, and those that do not materially affect the basic and novel characteristic(s) of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising”. The term “consisting of and variations thereof means limited to” unless expressly specified otherwise. In certain territories, the term “comprising an active ingredient consisting of” may be used in place of “consisting essentially”.

The term “about” in relation to a numerical value x means, for example, x±10%, x±5%, x±4%, x±3%, x±2%, x±1%.

Where methods refer to steps of administration, for example as (a), (b), (c), etc., these are intended to be sequential, i.e., step (c) follows step (b) which is preceded by step (a). Antibodies will generally be specific for their target, i.e., they will have a higher affinity for the target than for an irrelevant control protein, such as bovine serum albumin. Thus, unless specifically stated, a process comprising a step of mixing two or more components does not require any specific order of mixing. Thus, components can be mixed in any order. Where there are three components then two components can be combined with each other, and then the combination may be combined with the third component, etc.

Antibodies will generally be specific for their target. Thus, they will have a higher affinity for the target than for an irrelevant control protein, such as bovine serum albumin.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 54. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 55. However, with regard to fragments sequence identity will be measured by reference to the longest sequence. By way of non-limiting explanation, whilst the sequence of a fragment 10 amino acids long might be identical to a portion of a full-length sequence 100 amino acids long, the sequence identity based on the length of the longest sequence will be 10% (not 100% when calculated by reference to the shortest sequence).

MODES FOR CARRYING OUT THE INVENTION

### Example 1

### Preparation of Polypeptides

### Expression Plasmid and Recombinant Strain

Genes encoding the proteins of interest and a His-tag in C-term were cloned into pET24b(+) expression vector (Novagen) using the NdeI/XhoI restriction sites by means of standard procedures. Final constructs were generated by the transformation of E. coli B834(DE3) strain with the appropriate recombinant expression vector according to standard method with CaCl2-treated cells (Hanahan D. « Plasmid transformation by Simanis.» In Glover, D. M. (Ed), DNA cloning. IRL Press London. (1985): p. 109-135.).

### Host Strain

B834(DE3): Protease-deficient and methionine auxotroph strain. Strains having the designation (DE3) are lysogenic for a λ prophage that contains an IPTG inducible T7 RNA polymerase. λ DE3 lysogens are designed for protein expression from pET vectors. This strain is also deficient in the lon and ompT proteases. Genotype: B834::DE3 strain, F-ompT hsdSB(rB-mB-) gal dcm met (DE3)

### RTX Fragment (SEQ ID NO: 8)

A recombinant RTX fragment corresponding with residue 985 to residue 1681 of the wild-type adenylate cyclase was prepared as described below. The protein was his-20 tagged (GGHHHHHH) and secreted. The sequence contained the heterologous signal peptide coming from NspA (an outer membrane protein of Neisseria meningitidis). Two additional amino acid coming from this NspA were included to optimise signal peptide cleavage.

### Expression of the Recombinant Proteins—RTX Fragment

E. coli transformants were stripped from agar plate and used to inoculate LBT broth supplemented with 1% (w/v) glucose and kanamycin (50 μg/m1) to obtain optical density at 620 nm (O.D₆₂₀ₙₘ) between 0.1-0.2. Cultures were incubated overnight at 37° C. at a stiffing speed of 250 rpm. Overnight cultures were diluted to 1:20 in LBT medium containing kanamycin (50 μg/ml) and grown at 37° C., 250 rpm until O.D.620nm reached 0.5-0.8.

At an O.D.₆₂₀ₙₘ around 0.5-0.8, protein expression was induced by addition of 1 mM isopropyl β-D-1-thiogalactopyranoside and incubated 3 h at 37° C., 250 rpm. O.D₆₂₀ₙₘ were evaluated after 3h and cultures were centrifuged at 14 000 rpm for 15 minutes. Pellets were frozen at -20° C. separately.

### Purification of Polypeptide Fragments—RTX Fragment

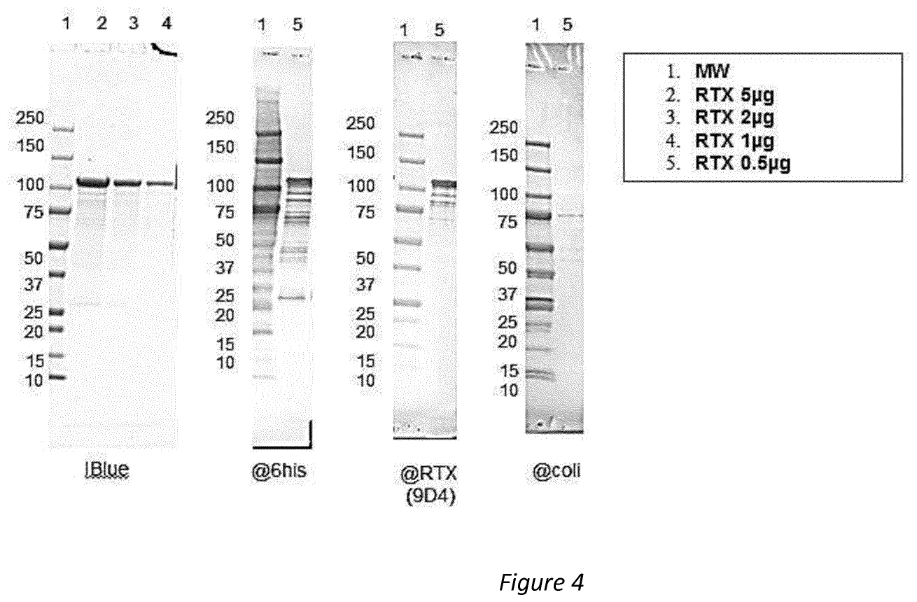

The bacterial pellets were re-suspended in lysis buffer (50 mM Tris pH 8.3, 300 mM NaCl, 10% glycerol, 2 mM CaCl₂, 2 mM Tris(2-carboxyethyl)phosphine)) (TCEP) (Sigma, St. Louis, Mo.) implemented with protease inhibitors (Complete without EDTA) (Roche Applied Science, Indianapolis, Ind.). Bacteria were lysed by French Press (3 times) at 1200 PSI and pelleted by centrifugation at 15 000 g for 60 min at 4° C. Ammonium sulfate (18% final) was added to the supernatant and rocked at room temperature for 20 min. The solution was centrifuged at 15 000 g for 15 min at room temperature. The supernatant was loaded using an AKTA™ Avant purification system onto a 5 ml His trap HPl l(GE Healthcare, Piscataway, N.J.) prequilibrated in buffer A (20 mM HEPES pH 7.6, 500 mM NaCl, 20 mM imidazole, 10% glycerol, 2 mM CaCl₂, 2 mM TCEP). The column was then washed by 5 column volume (CV) of buffer A followed by 6 CV of buffer A. The protein was eluted using 3CV of buffer B (20 mM HEPES pH 7.6, 500 mM NaCl, 500 mM imidazole, 10% glycerol, 2 mM CaCl₂, 2 mM TCEP). The fractions containing the protein of interest were pooled together and loaded onto a SUPERDEX™ 200 26/60 (GE Healthcare, Piscataway, N.J.). The protein was eluted with 1.5CV of buffer C B (20 mM HEPES pH 7.6, 150 mM NaCl, 500 mM imidazole, 5% glycerol, 2 mM CaCl₂, 1 mM TCEP). The purity of the protein was assessed by sodium dodecyl sulfate-polyacrylamide gel electrophoresis (SDS-PAGE). Fractions containing the antigen was selected and pooled together on the basis of purity by SDS-PAGE. Finally, the proteins were sterile filtered and stored at -80° C. Protein concentration was determined using the RC DCTM (reducing agent and detergent compatible) protein assay (Biorad, Hercules, CA) (FIG. 4).

### Expression of the Recombinant Proteins—AC Domain Fragments

E. coli transformants were stripped from agar plate and used to inoculate LBT broth supplemented with 1% (w/v) glucose and kanamycin (50 μg/ml) to obtain O.D.₆₂₀ₙₘ between 0.1-0.2. Cultures were incubated overnight at 37° C. at a stirring speed of 250 rpm. Overnight cultures were diluted to 1:20 in LBT medium containing kanamycin (50 μg/ml) and grown at 37° C., 250 rpm until O.D.620nm reached 0.5-0.8. At an O.D₆₂₀ₙₘ around 0.5-0.8, i) protein expression was induced by addition of 1 mM isopropyl β-D-1-thiogalactopyranoside and incubated 3 h at 37° C., 250 rpm ii) cultures were cooled down before inducing the expression of the recombinant protein by addition of 1 mM isopropyl β-D-1-thiogalactopyranoside and incubated overnight at 16° C., 250 rpm. After the 3 h/overnight inductions (around 16 hours), O.D₆₂₀ₙₘ were evaluated, cultures were centrifuged at 14 000 rpm for 15 minutes and pellets were frozen at −20° C. separately.

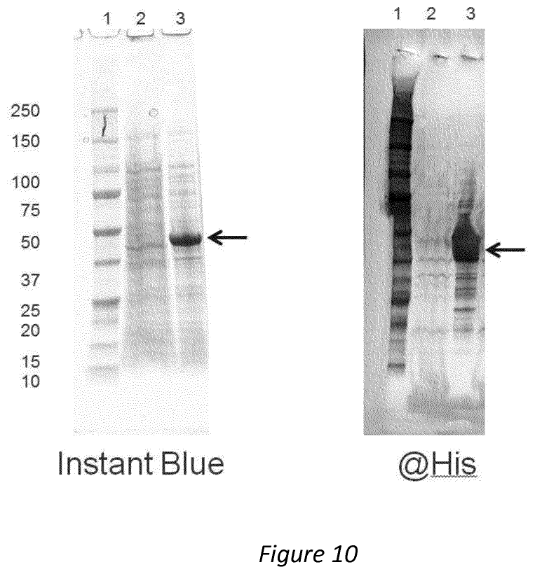

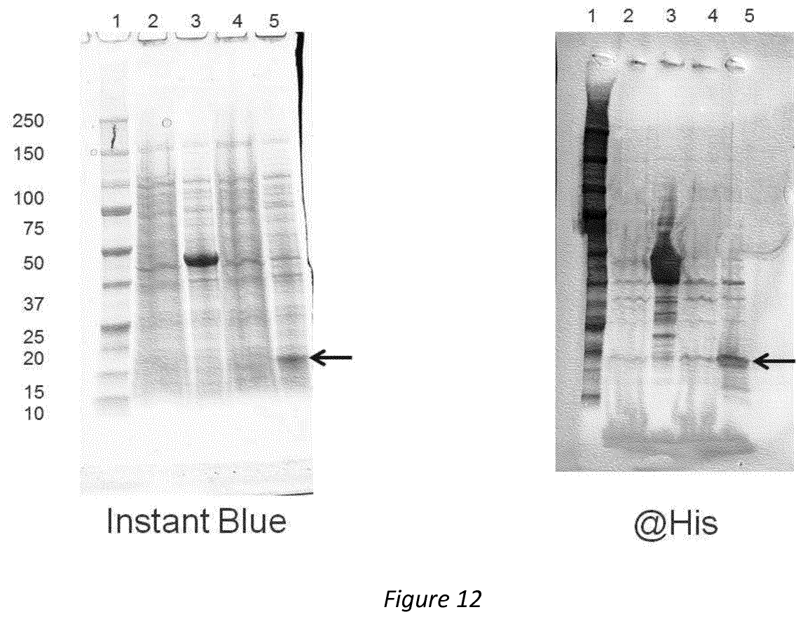

Expression of SEQ ID NO: 21 under the following conditions is shown in FIG. 10: E. coli strain: B834(DE3), IPTG concentration: 1 mM, Induction: 16° C., Overnight. (1) Molecular Weight Marker; (2) Non-induced; (3) Induced. Expression of SEQ ID NO: 22 under the following conditions is shown in FIG. 12: Expression conditions: E. coli strain: B834(DE3), IPTG concentration: 1 mM, Induction: 37° C., 3 h. (1) Molecular Weight Marker; (2) Non-induced; (3) Induced; (4) Non-induced; (5) Induced.

Purification of polypeptide Fragments—AC Domain Fragments

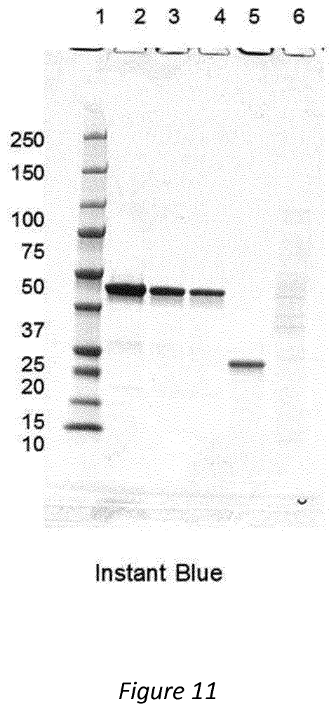

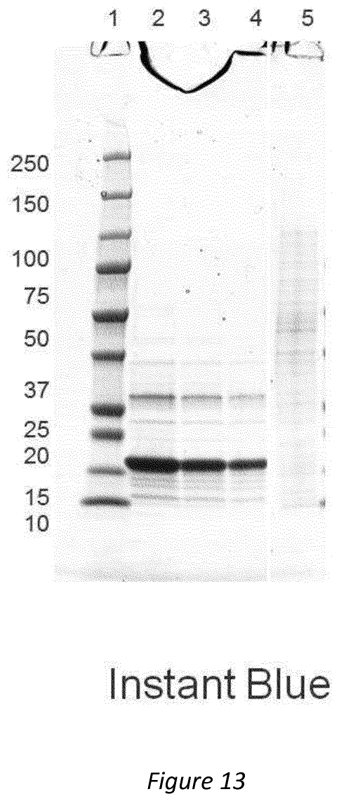

The bacterial pellets were re-suspended in lysis buffer (20 mM HEPES pH 7.6, 500 mM NaCl, 10% glycerol, 20 mM imidazole, 5 mM MgCl₂ (Sigma, St. Louis, Mo.) implemented with protease inhibitors (Complete without EDTA) (Roche Applied Science, Indianapolis, Ind.) and 125 units/ml of benzonase (Sigma, St. Louis, Mo.). Bacteria were lysed by French Press (3 times) at 1200 PSI and pelleted by centrifugation at 15 000g for 30 min at 4° C. The supernatant was loaded using an AKTA™ Avant purification system onto a 5 ml His trap HP (GE Healthcare, Piscataway, N.J.) prequilibrated in buffer A (20 mM HEPES pH 7.6, 500 mM NaCl, 20mM imidazole, 10% glycerol). The column was then washed by 10 column volume (CV) of buffer A. The protein was eluted using 3CV of buffer B (20 mM HEPES pH 7.6, 500 mM NaCl, 500 mM imidazole, 10% glycerol). The fractions containing the protein of interest were pooled together and loaded onto a SUPERDEX™ 75 26/60 (GE Healthcare, Piscataway, N.J.). The protein was eluted with 1.5CV of buffer C B (20 mM HEPES pH 7.6, 150 mM NaCl, 5% glycerol). The purity of the protein was assessed by sodium dodecyl sulfate-polyacrylamide gel electrophoresis (SDS-PAGE). Fractions containing the antigen was selected and pooled together on the basis of purity by SDS-PAGE. Finally, the proteins were sterile filtered and stored at −80° C. Protein concentration was determined using the RC DCTM (reducing agent and detergent compatible) protein assay (Biorad, Hercules, Calif.). Purification results for SEQ ID NO: 21 are provided in FIG. 11. Purification results for SEQ ID NO: 22 are provided in FIG. 13.

### Example 2

### Formulation of RTX polypeptides with Al(OH)₃ or AS01

Six formulations were prepared:
Formulation 1 RTX 500 μg/mL+AS01E Ca 2 mM (Folded RTX)
Formulation 2 RTX 50 μg/mL+AS01E Ca 2 mM (Folded RTX)
Formulation 3 RTX 500 μg/mL+AS01E Ca 2 mM+5 mM EGTA(Unfolded RTX)
Formulation 4 RTX 50 μg/mL+AS01E Ca 2 mM+5 mM EGTA(Unfolded RTX)
Formulation 5 RTX 500 μg/mL+AL(OH)3Ca 2 mM
Formulation 6 RTX 50 μg/mL+AL(OH)3 Ca 2 mM

### [NB: RTX Refers to the RTX Polypeptide Fragment]

Each formualtion contained the following constituents:

| | | 1 | 2 | 3 | 4 | 5 | 6 |
|---|---|---|---|---|---|---|---|
| PO4 | mM | 3.50 | 8.65 | 3.50 | 8.65 | 0.00 | 7.65 |
| NaCl | mM | 127.87 | 115.49 | 127.87 | 115.49 | 93.87 | 116.49 |
| TCEP | mM | 0.63 | 0.06 | 0.63 | 0.06 | 0.63 | 0.06 |
| HEPES | mM | 12.52 | 1.25 | 12.52 | 1.25 | 12.52 | 1.25 |
| Glycérol | % | 3.13 | 0.31 | 3.13 | 0.31 | 3.13 | 0.31 |
| CaCl2 | mM | 2.00 | 2.00 | 2.00 | 2.00 | 2.00 | 2.00 |
| EGTA | mM | 0.00 | 0.00 | 5.00 | 5.00 | 0.00 | 0.00 |
| AS01 | μg/ml | 50.00 | 50.00 | 50.00 | 50.00 | 0.00 | 0.00 |
| Al(OH)₃ | μg/ml | 0.00 | 0.00 | 0.00 | 0.00 | 1000.00 | 1000.00 |
| RTX | μg/ml | 500.00 | 50.00 | 500.00 | 50.00 | 500.00 | 50.00 |

To prepare 2000 uL of formulations 1 to 4, the RTX polypeptide fragments in formulation buffer (150 mM NaCl, 1 mM TCEP, 20 mM HEPES, 5% Glycerol, 2 mM CaCl₂, 563 mM NaCl) were mixed with CaCl₂ and PBS (pH7.4) and stirred for 5 to 30 minutes at room temperature. EGTA (also referred to as Egtazic acid or Ethylenebis(oxyethylenenitrilo)tetraacetic acid) was added to formulations 3 and 4 to a final concentration of 5 mM. In a final step, AS01 adjuvant was added and the formulation stirred for a further 5 to 30 minutes at room temperature. To prepare 200 uL of formulations 5 and 6, the RTX polypeptide fragments in formulation buffer was mixed with CaCl₂ and PBS. Al(OH)₃ was added in a final step before stirring for 60 to 120 minutes at room temperature.

### Adsorption of Polypeptide Fragments on Al(OH)₃

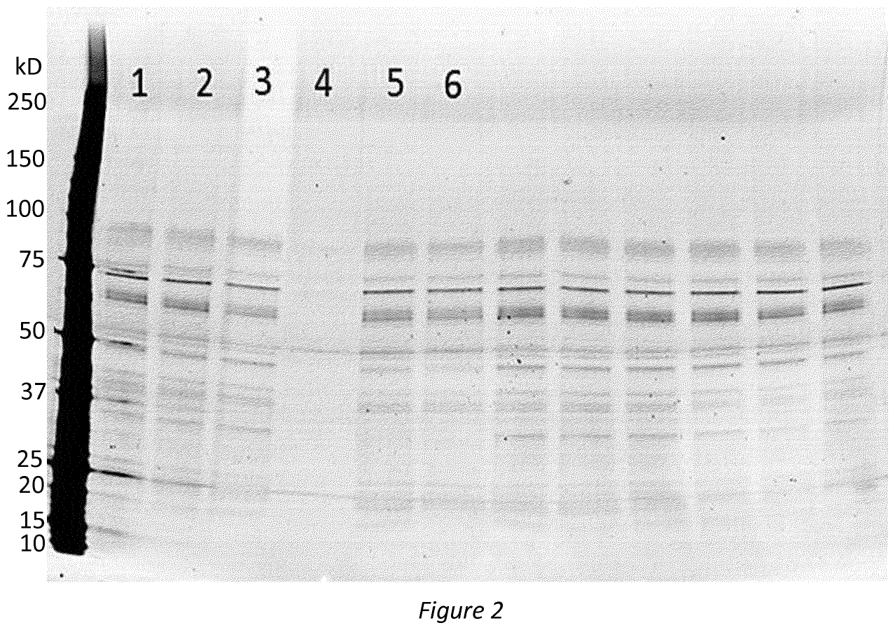

Absence of polypeptide fragment in supernatant of formulation with Al(OH)₃ suggests that the polypeptide fragment is well adsorbed (FIG. 2). These results were confirmed in NanoDSF and UPLC-SEC.

Compatibility of AS01 with Polypeptide Fragments and/or EGTA

AS01 particle size was preserved and there was an absence of hemolysis in all conditions.

Conformation of Polypeptide Fragments in Presence of AS01 and/or EGTA

In presence of AS01 or AS01 buffer fragment peak (NanoDSF) is preserved. This data confirms that the secondary structure of the Adenylate cyclase fragments are preserved (folded state). In presence of EGTA there is a loss of the fragment peak and typical Tm is observed even in presence of ASO1. This data indicates that the secondary structure of the fragments is not preserved without presence of Ca++. When formulated with Al(OH)₃, loss of the fragment peak is observed because the polypeptide fragment is adsorbed at the surface.

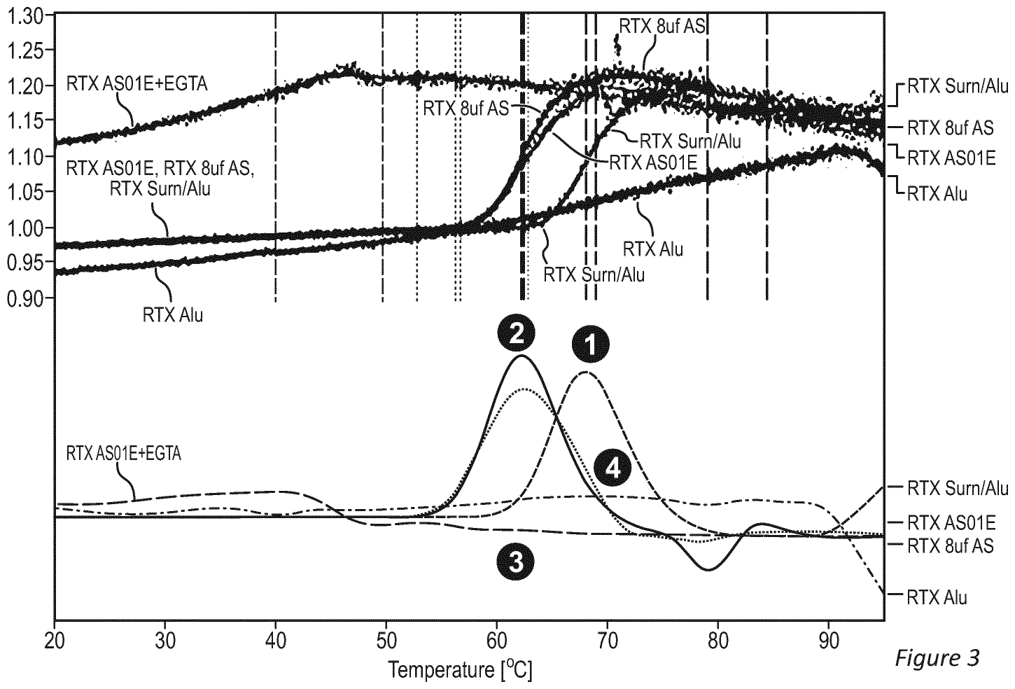

Results are provided in FIG. 3.

### Example 3

### Evaluation of immunogenicity of RTX fragments in Folded/Unfolded State

Six groups of 15 female mice (BALB/cOlaHsd aged 6 weeks) were immunized intramuscularly with 50 μL of each vaccine formulation on days 1, 14 and 28 as indicated in Table 1. Partial bleed was performed on day 28 (14PII) and final bleed on day 42 (14PIII). Table 1:

| | | | Dose of | |
|---|---|---|---|---|
| Gr. | N | Vaccine | RTX | Calcium |
| 1 | 15 | RTX + | 25 μg | 2 mM |
| 2 | 15 | AS01E | 2.5 μg | |
| 3 | 15 | | 25 μg | 2 mM + 5 mM |
| 4 | 15 | | 2.5 μg | EGTA |
| 5 | 15 | RTX + | 25 μg | 2 mM |
| 6 | 15 | AL(OH)₃ | 2.5 μg | |

### Adenylate Cyclase Toxin Sero-Neutralisation Assay

Adenylate cyclase Toxin cytotoxicity neutralization assay was performed as described in Eby et al. (Clinical and Vaccine Immunology, January 2017 Volume 24 Issue 1, pages 1-10) but with minor modifications to the concentration of ACT:

TABLE 2

| | | | | 95% | 95% |
|---|---|---|---|---|---|
| | | | | CI | CI |
| | | | GMR | Lower | Upper |
| TP | Group (A) | Group (B) | (A/B) | Limit | Limit |
| PIII | RTX 25 μg AS01E Ca | RTX 25 μg Al(OH)3 | 1.84 | 1.38 | 2.46 |
| | 2 mM | Ca 2 mM | | | |
| PIII | RTX 2.5 μg AS01E Ca | RTX 2.5 μg Al(OH)3 | 2.26 | 1.32 | 3.88 |
| | 2 mM | Ca 2 mM | | | |
| PIII | RTX 25 μg AS01E Ca | RTX 25 μg AS01E | 1.24 | 0.94 | 1.65 |
| | 2 mM + 5 mM EGTA | Ca 2 mM | | | |
| PIII | RTX 2.5 μg AS01E Ca | RTX 2.5 μg AS01E | 1.27 | 0.96 | 1.67 |
| | 2 mM + 5 mM EGTA | Ca 2 mM | | | |
| PIII | RTX 25 μg AS01E Ca | RTX 25 μg Al(OH)3 | 2.29 | 1.64 | 3.19 |
| | 2 mM + 5 mM EGTA | Ca 2 mM | | | |
| PIII | RTX 2.5 μg AS01E Ca | RTX 2.5 μg Al(OH)3 | 2.87 | 1.71 | 4.82 |
| | 2 mM + 5 mM EGTA | Ca 2 mM | | | |

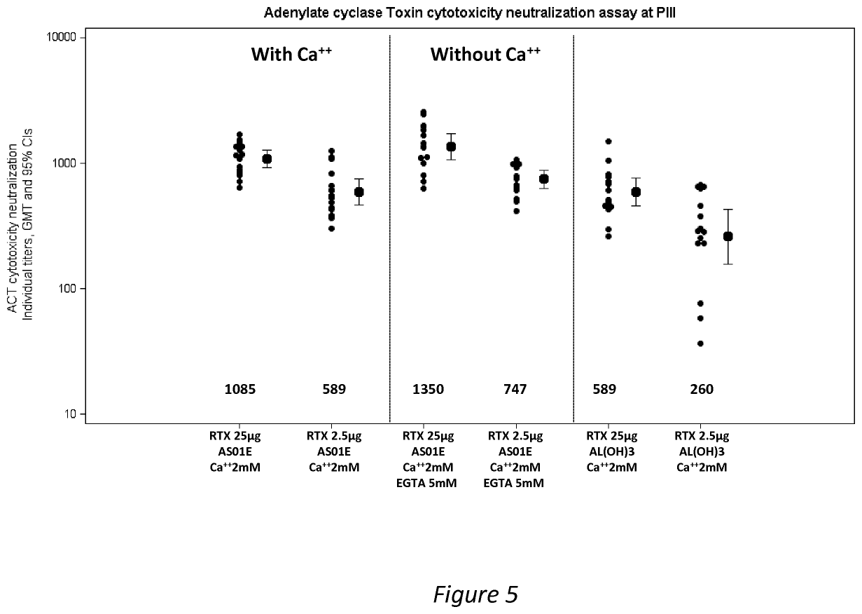

Equivalence was observed between AS01E (an adjuvant System containing MPL, QS21 and liposomes (25 μg MPL and 25 μg QS21)) adjuvanted formulations both with and without Ca++ at each dose. However, the response to formulations adjuvanted with AL(OH)₃ was lower when compared to other formulations at each dose (FIG. 5).

### Example 4

### Combination of RTX Fragments With DTaP Vaccines

All acellular pertussis (referred to as Pa or aP) vaccines evaluated in this study were from GlaxoSmithKline Biologicals (GSK, Rixensart, Belgium). The amount of the different B. pertussis antigen components per dose used in four groups is shown below in Table 3:

| | | | | Dose of Antigen per mouse | | | | | | | Challenge |
|---|---|---|---|---|---|---|---|---|---|---|---|
| | | | Route/ | RTX | DT | TT | PT | FHA | PRN | Injections | 18323 |
| Group | No. | Vaccine | Vol. | (μg) | (Lf) | (Lf) | (μg) | (μg) | (μg) | (Days) | Bp strain (Day) |
| 1 | 20 | Control | — | — | — | — | — | — | — | 0, 21 | 29 |
| 2 | 20 | Infanrix | SC- | — | 6.25 | 2.5 | 6.25 | 6.25 | 2 | 0, 21 | 29 |
| | | DTPa | 125 μl | | | | | | | | |
| | | (1/4 HD) | | | | | | | | | |
| 3 | 20 | Infanrix | SC- | — | 6.25 | 2.5 | 6.25 | 6.25 | 0.05 | 0, 21 | 29 |
| | | DTPa | 125 μl | | | | | | | | |
| | | (PT + FHA - | | | | | | | | | |
| | | 1/4th HD, | | | | | | | | | |
| | | PRN | | | | | | | | | |
| | | 1/160 HD) | | | | | | | | | |
| 4 | 20 | Infanrix | SC- | 25 | 6.25 | 2.5 | 6.25 | 6.25 | 0.05 | 0, 21 | 29 |
| | | DTPa | 125 μl | | | | | | | | |
| | | (PT + FHA - | | | | | | | | | |
| | | 1/4th HD, | | | | | | | | | |
| | | PRN | | | | | | | | | |
| | | 1/160 | | | | | | | | | |
| | | HD) | | | | | | | | | |

*HD = Human Dose; SC = Subcutaneous

### Animal Model

All experiments and assays were performed at GSK laboratories (Rixensart, Belgium) in accordance with European Directive 2010/63/EU and the GlaxoSmithKline Biologicals' policy on the care, welfare and treatment of animals. Animals had free access to water and a maintenance diet. Consistent with the Association for Assessment and Accreditation of Laboratory Animal Care (AAALAC) global enrichment program, the environment included nesting material (Envirodry), and social housing was applied.

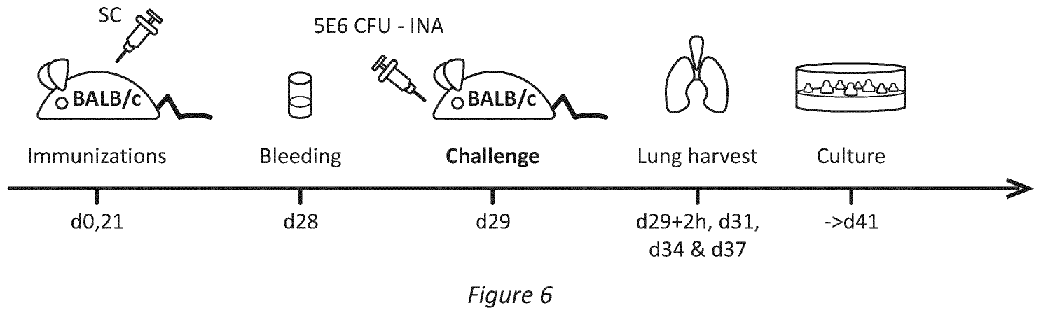

The in vivo mouse B. pertussis lung clearance assay is based on the analysis of the lung invasion by Bordetella strains following standard sublethal intranasal challenge of vaccinated mice [20]. Groups of 18 or 20 BALB/c OlaHsd mice (females, 6 weeks old) were given two doses of vaccine at 3-week intervals. Blood samples were collected on day 28. One week after the booster (day 29), mice were challenged by instillation of 50 μl of bacterial suspension (equivalent to a total of approximately 5×10⁶ colony forming units [CFU]) into the left nostril under light anaesthesia with isoflurane (2.5%). For the intranasal challenge models, bacterial suspensions of B. pertussis grown on Bordet-Gengou agar plates (BGA) and in Stainer-Scholte liquid medium as described above, were diluted in Stainer-Scholte medium to provide a challenge inoculum of approximately 1×10⁸ CFU/ml for a sublethal challenge. A 50 μl aliquot of bacterial suspension was slowly administered into the nostril using a micropipette to be immediately aspirated by the animals' respiration. After sublethal challenge, three or five infected mice were sacrificed by anaesthesia 2 hours, 2 days, 5 days and 8 days after exposure (designated days 29+2 hours, D31, D34 and D37). The lungs were removed and homogenized in 2 ml casaminoacid (1%) buffered saline with tissue grinders. 10-fold serial dilutions of the homogenates were plated on BGA and incubated at 36-37 ° C. for 4-6 days before counting of CFU. The log10 weighted mean number of CFU per lung (CFUw/lung) and the standard deviation were calculated for each time point. A schematic of the immunisation schedule is provided in FIG. 6.

Serology Analysis for B. pertussis antigens:
Pertussis Toxoid (PT), Filamentous hemagglutinin (FHA), Pertactin (PRN) and Adenylate cyclase (ACT)

Sera from all mice were individually collected seven days after the second immunization (day28—the day before challenge) and tested for the presence of anti-PT, -FHA, and PRN IgG antibodies according to the following protocols:

PT, PRN and FHA serology protocols

96-well plates were coated with FHA (2 μg/ml), PT (2 μg/ml) or PRN (3 μg/m1) in a carbonate-bicarbonate buffer (50 mM) and incubated overnight at 4° C. After the saturation step with the PBS-BSA 1% buffer, mouse sera were diluted at 1/100 in PBS-BSA 0.2% Tween 0.05% and serially diluted in the wells from the plates (12 dilutions, step 1/2). An anti-mouse IgG coupled to the peroxidase was added (1/3000 final dilution). Colorimetric reaction was observed after the addition of the peroxidase substrate (OPDA), and stopped with HCl M before reading by spectrophotometry (wavelengths: 490-620 nm). For each serum tested and standard added on each plate, a 4-parameter logistic curve was fit to the relationship between the OD and the dilution (Softmaxpro). This allowed the derivation of each sample titer expressed in STD titers.

### ACT Serology Protocol

96-well plates were coated with ACT (0.3 μg/ml) in a carbonate-bicarbonate buffer (50 mM) and incubated overnight at 4° C. After the saturation step with the buffer TR020 (saturation buffer ELISA—NBC Serum 4% V/V), mouse sera were diluted at 1/100 in TR020 and serially diluted in the wells from the plates (12 dilutions, step 1/2). An anti-mouse IgG coupled to the peroxidase was added (1/1000 final dilution). Colorimetric reaction was observed after the addition of the peroxidase substrate (OPDA), and stopped with HCL 1M before reading by spectrophotometry (wavelengths: 490-620 nm). For each serum tested and standard added on each plate, a 4-parameter logistic curve was fit to the relationship between the OD and the dilution (Softmaxpro). This allowed the derivation of each sample titer expressed in STD titers.

### Adenylate Cyclase Toxin Sero-Neutralization Assays

The toxin neutralization assay is based on the cytotoxicity of ACT toward J774.A1 macrophage-like cells. The J774.A1 cells in D-MEM (250,000 cells in 50 μl) were seeded in each well of a 96-well plate and let overnight at 37° C. with 5% CO₂ to allow attachment. The following day, mAb 3D1 (Kerafast) and pools of the collected sera were serially diluted in D-MEM and mixed with ACT before transfer to the cells at a final ACT concentration of 0.8 or 2 or 3 μg/ml. After 2h incubation at 37° C., the Cell Counting Kit-8 (CCK-8, Dojindo) was used to determine cell viability by colorimetry based on the reduction of the water-soluble tetrazolium salt (WST-8) by dehydrogenases in live cells, generating a yellow-color formazan dye. For each standard (mAb 3D1) and pooled sera tested on each plate, a 4-parameter logistic curve was fit to the relationship between the OD and the dilutions (Softmaxpro). Titers were expressed in terms of “MidPoint” derived from the “Midpoint” of the standard of each plate.

### Statistical Methods

The distributions of mean of number of colony-forming unit (CFU) is assumed to be lognormal. The statistical method was an Analysis of Variance (ANOVA) on the log10 values with 2 factors (group and day) using a heterogeneous variance model i.e identical variances were not assumed for the different levels of the factors combinations. The interaction between formulation and dose was not included in the model (assumption: non-significant interaction). As exploratory analysis, no adjustment will be performed. This model is used to estimate geometric means and their 95% CIs as well as geometric mean ratios and their 95% CIs.

The distribution of the IgG titres is assumed to be lognormal. The statistical method is an

Analysis of Variance (ANOVA) by antigen on the log10 values with 1 factor (group). This model was used to estimate geometric means and their 95% CIs as well as geometric mean ratios and their 95% CIs.

Anti-PRN antibodies were detected in all conditions, confirming successful vaccination.

As expected, levels of anti-PRN antibodies were lower in mice that received 1/80 HD versus ¼ HD.

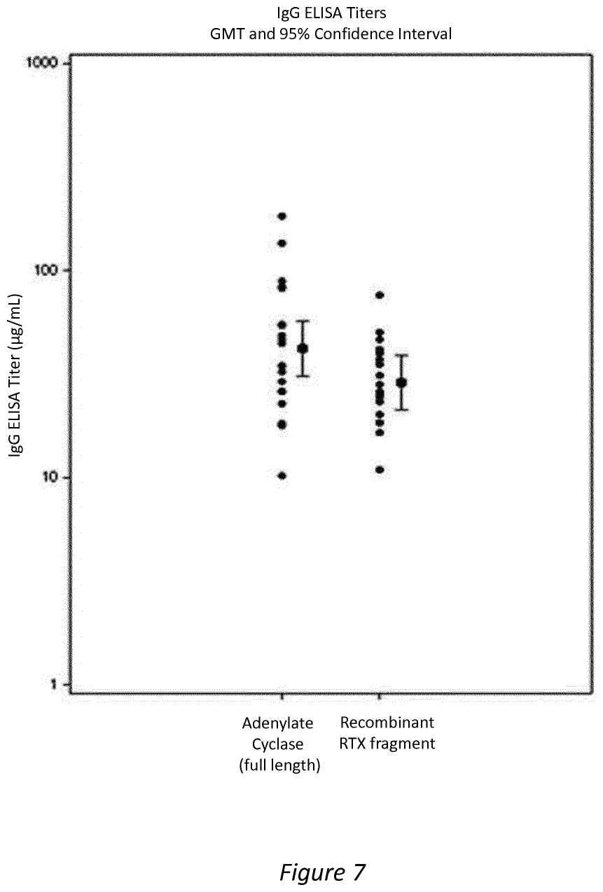

As shown in FIG. 7, immunisation with the RTX fragments is able to induce levels of anti-ACT IgG to levels similar to those obtained with non-detoxified full length adenylate cyclase.

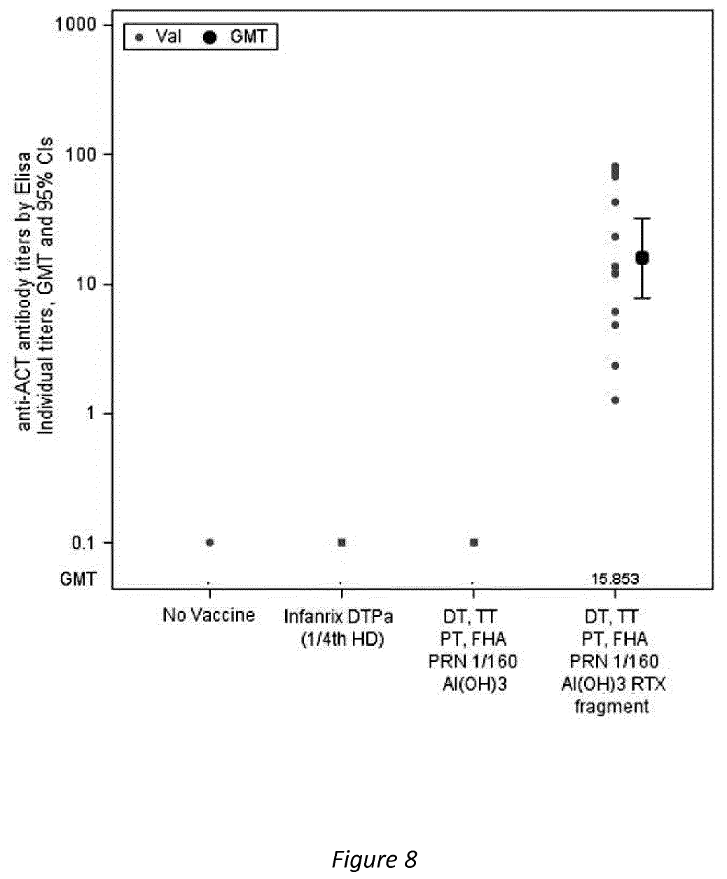

Individual antibody titers (anti-ACT IgG) were measured by ELISA at day 28 (FIG. 8). The RTX fragment induced high levels of anti-ACT IgG.

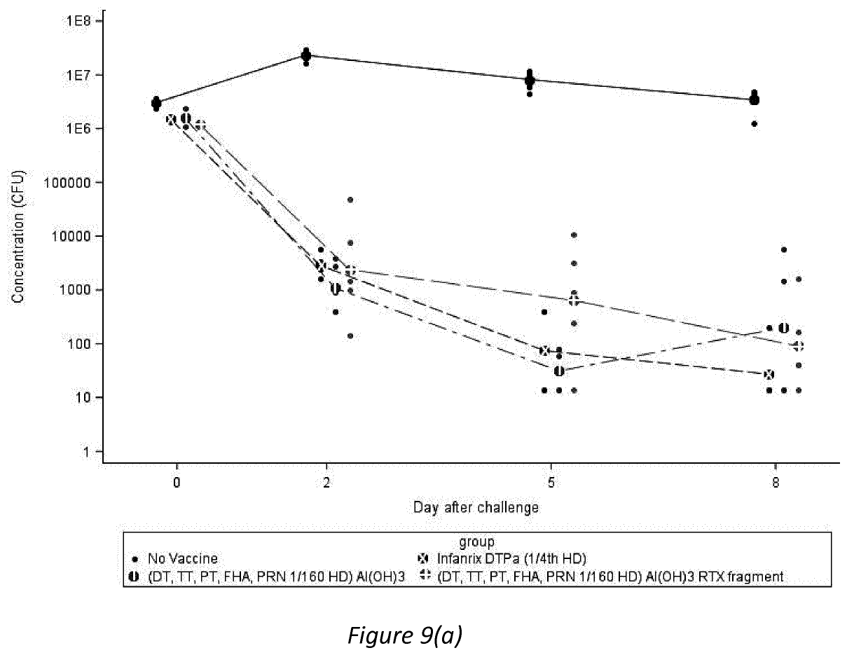

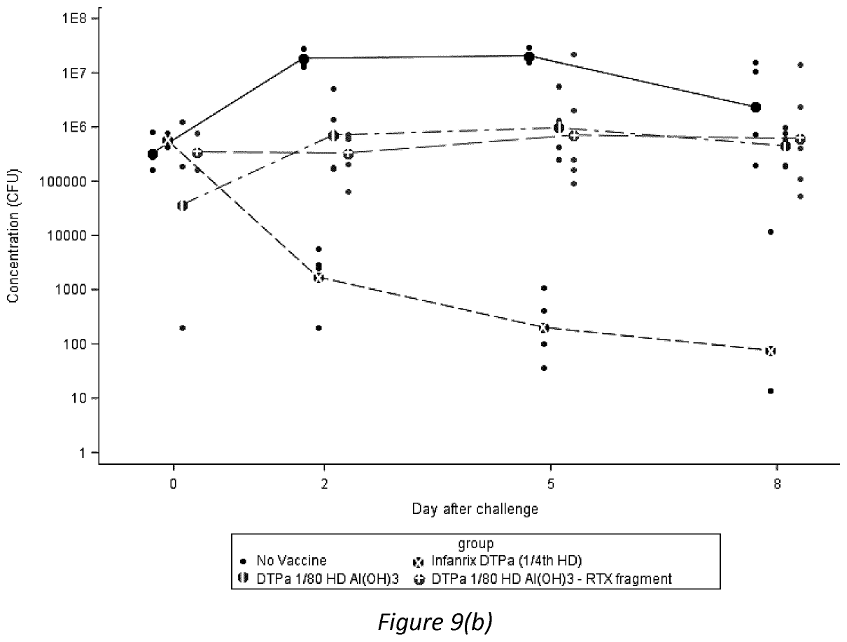

The protective efficacy against B. pertussis intranasal challenge induced by the different vaccines is shown in FIGS. 9(a) and 9(b).

Investigated formulations, including the Infanrix ¼th HD group (positive control) reduced the number of CFU with respect to the unvaccinated group. High significant differences were observed between Infanrix ¼th HD group (positive control) and DTPa 1/80HD with/without the RTX fragment. However, whilst the RTX fragment is clearly immunogenic, reduces the number of CFU with respect to the unvaccinated group and is capable of inducing antibodies, in the experimental model used, no discernible improvement on protective efficacy was observed when the RTX fragment was combined with DTPa as compared to DTPa alone ( 1/80HD). Thus, the RTX fragment may be useful for inclusion in a DTPa vaccine from the perspective of reducing colonisation.

SEQUENCES

SEQ ID NO: 1-adenylate cyclase Bordetella pertussis (strain Tohama I/
ATCCBAA-589
MQQSHQAGYANAADRESGIPAAVLDGIKAVAKEKNATLMFRLVNPHSTSLIAEGVATK
GLGVHAKSSDWGLQAGYIPVNPNLSKLFGRAPEVIARADNDVNSSLAHGHTAVDLTLS
KERLDYLRQAGLVTGMADGVVASNHAGYEQFEFRVKETSDGRYAVQYRRKGGDDFE
AVKVIGNAAGIPLTADIDMFAIMPHLSNFRDSARSSVTSGDSVTDYLARTRRAASEATG
GLDRERIDLLWKIARAGARSAVGTEARRQFRYDGDMNIGVITDFELEVRNALNRRAHA
VGAQDVVQHGTEQNNPFPEADEKIFVVSATGESQMLTRGQLKEYIGQQRGEGYVFYEN
RAYGVAGKSLFDDGLGAAPGVPSGRSKFSPDVLETVPASPGLRRPSLGAVERQDSGYDS
LDGVGSRSFSLGEVSDMAAVEAAELEMTRQVLHAGARQDDAEPGVSGASAHWGQRAL
QGAQAVAAAQRLVHAIALMTQFGRAGSTNTPQEAASLSAAVFGLGEASSAVAETVSGF
FRGSSRWAGGFGVAGGAMALGGGIAAAVGAGMSLTDDAPAGQKAAAGAEIALQLTG
GTVELASSIALALAAARGVTSGLQVAGASAGAAAGALAAALSPMEIYGLVQQSHYADQ
LDKLAQESSAYGYEGDALLAQLYRDKTAAEGAVAGVSAVLSTVGAAVSIAAAASVVG
APVAVVTSLLTGALNGILRGVQQPIIEKLANDYARKIDELGGPQAYFEKNLQARHEQLA
NSDGLRKMLADLQAGWNASSVIGVQTTEISKSALELAAITGNADNLKSVDVFVDRFVQ
GERVAGQPVVLDVAAGGIDIASRKGERPALTFITPLAAPGEEQRRRTKTGKSEFTTFVEI
VGKQDRWRIRDGAADTTIDLAKVVSQLVDANGVLKHSIKLDVIGGDGDDVVLANASRI
HYDGGAGTNTVSYAALGRQDSITVSADGERFNVRKQLNNANVYREGVATQTTAYGKR
TENVQYRHVELARVGQLVEVDTLEHVQHIIGGAGNDSITGNAHDNFLAGGSGDDRLD
GGAGNDTLVGGEGQNTVIGGAGDDVFLQDLGVWSNQLDGGAGVDTVKYNVHQPSEE
RLERMGDTGIHADLQKGTVEKWPALNLFSVDHVKNIENLHGSRLNDRIAGDDQDNEL
WGHDGNDTIRGRGGDDILRGGLGLDTLYGEDGNDIFLQDDETVSDDIDGGAGLDTVDY
SAMIHPGRIVAPHEYGFGIEADLSREWVRKASALGVDYYDNVRNVENVIGTSMKDVLIG
DAQANTLMGQGGDDTVRGGDGDDLLFGGDGNDMLYGDAGNDTLYGGLGDDTLEGG
AGNDWFGQTQAREHDVLRGGDGVDTVDYSQTGAHAGIAAGRIGLGILADLGAGRVDK
LGEAGSSAYDTVSGIENVVGTELADRITGDAQANVLRGAGGADVLAGGEGDDVLLGG
DGDDQLSGDAGRDRLYGEAGDDWFFQDAANAGNLLDGGDGRDTVDFSGPGRGLDAG
AKGVFLSLGKGFASLMDEPETSNVLRNIENAVGSARDDVLIGDAGANVLNGLAGNDVL
SGGAGDDVLLGDEGSDLLSGDAGNDDLFGGQGDDTYLFGVGYGHDTIYESGGGHDTIR
INAGADQLWFARQGNDLEIRILGTDDALTVHDWYRDADHRVEIIHAANQAVDQAGIEK
LVEAMAQYPDPGAAAAAPPAARVPDTLMQSLAVNWR
SEQ ID NO: 2-Adenylate cyclase fragment without His tag
TENVQYRHVELARVGQLVEVDTLEHVQHIIGGAGNDSITGNAHDNFLAGGSGDDRLDG
GAGNDTLVGGEGQNTVIGGAGDDVFLQDLGVWSNQLDGGAGVDTVKYNVHQPSEER
LERMGDTGIHADLQKGTVEKWPALNLFSVDHVKNIENLHGSRLNDRIAGDDQDNELW
GHDGNDTIRGRGGDDILRGGLGLDTLYGEDGNDIFLQDDETVSDDIDGGAGLDTVDYS
AMIHPGRIVAPHEYGFGIEADLSREWVRKASALGVDYYDNVRNVENVIGTSMKDVLIG
DAQANTLMGQGGDDTVRGGDGDDLLFGGDGNDMLYGDAGNDTLYGGLGDDTLEGG
AGNDWFGQTQAREHDVLRGGDGVDTVDYSQTGAHAGIAAGRIGLGILADLGAGRVDK
LGEAGSSAYDTVSGIENVVGTELADRITGDAQANVLRGAGGADVLAGGEGDDVLLGG
DGDDQLSGDAGRDRLYGEAGDDWFFQDAANAGNLLDGGDGRDTVDFSGPGRGLDAG
AKGVFLSLGKGFASLMDEPETSNVLRNIENAVGSARDDVLIGDAGANVLNGLAGNDVL
SGGAGDDVLLGDEGSDLLSGDAGNDDLFGGQGDDTYLFGVGYGHDTIYESGGGHDTIR
INAGADQLWFARQGNDLEIRILGTDDALTVHDWYRDADHRVEIIHAANQAVDQAGIEK
LVEAMAQYPDP
SEQ ID NO: 3-Adenylate cyclase fragment without His tag
TENVQYRHVELARVGQLVEVDTLEHVQHIIGGAGNDSITGNAHDNFLAGGSGDDRLDG
GAGNDTLVGGEGQNTVIGGAGDDVFLQDLGVWSNQLDGGAGVDTVKYNVHQPSEER
LERMGDTGIHADLQKGTVEKWPALNLFSVDHVKNIENLHGSRLNDRIAGDDQDNELW
GHDGNDTIRGRGGDDILRGGLGLDTLYGEDGNDIFLQDDETVSDDIDGGAGLDTVDYS
AMIHPGRIVAPHEYGFGIEADLSREWVRKASALGVDYYDNVRNVENVIGTSMKDVLIG
DAQANTLMGQGGDDTVRGGDGDDLLFGGDGNDMLYGDAGNDTLYGGLGDDTLEGG
AGNDWFGQTQAREHDVLRGGDGVDTVDYSQTGAHAGIAAGRIGLGILADLGAGRVDK
LGEAGSSAYDTVSGIENVVGTELADRITGDAQANVLRGAGGADVLAGGEGDDVLLGG
DGDDQLSGDAGRDRLYGEAGDDWFFQDAANAGNLLDGGDGRDTVDFSGPGRGLDAG
AKGVFLSLGKGFASLMDEPETSNVLRNIENAVGSARDDVLIGDAGANVLNGLAGNDVL
SGGAGDDVLLGDEGSDLLSGDAGNDDLFGGQGDDTYLFGVGYGHDTIYESGGGHDTIR
INAGADQLWFARQGNDLEIRILGTDDALTVHDWYRDADHRVEIIHAANQAVDQAGIEK
LVEAMAQYPDPG
SEQ ID NO: 4-Adenylate cyclase fragment without His tag
TENVQYRHVELARVGQLVEVDTLEHVQHIIGGAGNDSITGNAHDNFLAGGSGDDRLDG
GAGNDTLVGGEGQNTVIGGAGDDVFLQDLGVWSNQLDGGAGVDTVKYNVHQPSEER
LERMGDTGIHADLQKGTVEKWPALNLFSVDHVKNIENLHGSRLNDRIAGDDQDNELW
GHDGNDTIRGRGGDDILRGGLGLDTLYGEDGNDIFLQDDETVSDDIDGGAGLDTVDYS
AMIHPGRIVAPHEYGFGIEADLSREWVRKASALGVDYYDNVRNVENVIGTSMKDVLIG
DAQANTLMGQGGDDTVRGGDGDDLLFGGDGNDMLYGDAGNDTLYGGLGDDTLEGG
AGNDWFGQTQAREHDVLRGGDGVDTVDYSQTGAHAGIAAGRIGLGILADLGAGRVDK
LGEAGSSAYDTVSGIENVVGTELADRITGDAQANVLRGAGGADVLAGGEGDDVLLGG
DGDDQLSGDAGRDRLYGEAGDDWFFQDAANAGNLLDGGDGRDTVDFSGPGRGLDAG
AKGVFLSLGKGFASLMDEPETSNVLRNIENAVGSARDDVLIGDAGANVLNGLAGNDVL
SGGAGDDVLLGDEGSDLLSGDAGNDDLFGGQGDDTYLFGVGYGHDTIYESGGGHDTIR
INAGADQLWFARQGNDLEIRILGTDDALTVHDWYRDADHRVEIIHAANQAVDQAGIEK
LVEAMAQYPDPGG
SEQ ID NO: 5-Adenylate cyclase plus His tag 1
TENVQYRHVELARVGQLVEVDTLEHVQHIIGGAGNDSITGNAHDNFLAGGSGDDRLDG
GAGNDTLVGGEGQNTVIGGAGDDVFLQDLGVWSNQLDGGAGVDTVKYNVHQPSEER
LERMGDTGIHADLQKGTVEKWPALNLFSVDHVKNIENLHGSRLNDRIAGDDQDNELW
GHDGNDTIRGRGGDDILRGGLGLDTLYGEDGNDIFLQDDETVSDDIDGGAGLDTVDYS
AMIHPGRIVAPHEYGFGIEADLSREWVRKASALGVDYYDNVRNVENVIGTSMKDVLIG
DAQANTLMGQGGDDTVRGGDGDDLLFGGDGNDMLYGDAGNDTLYGGLGDDTLEGG
AGNDWFGQTQAREHDVLRGGDGVDTVDYSQTGAHAGIAAGRIGLGILADLGAGRVDK
LGEAGSSAYDTVSGIENVVGTELADRITGDAQANVLRGAGGADVLAGGEGDDVLLGG
DGDDQLSGDAGRDRLYGEAGDDWFFQDAANAGNLLDGGDGRDTVDFSGPGRGLDAG
AKGVFLSLGKGFASLMDEPETSNVLRNIENAVGSARDDVLIGDAGANVLNGLAGNDVL
SGGAGDDVLLGDEGSDLLSGDAGNDDLFGGQGDDTYLFGVGYGHDTIYESGGGHDTIR
INAGADQLWFARQGNDLEIRILGTDDALTVHDWYRDADHRVEIIHAANQAVDQAGIEK
LVEAMAQYPDPHHHHHH
SEQ ID NO: 6-Adenylate cyclase plus His tag 2
TENVQYRHVELARVGQLVEVDTLEHVQHIIGGAGNDSITGNAHDNFLAGGSGDDRLDG
GAGNDTLVGGEGQNTVIGGAGDDVFLQDLGVWSNQLDGGAGVDTVKYNVHQPSEER
LERMGDTGIHADLQKGTVEKWPALNLFSVDHVKNIENLHGSRLNDRIAGDDQDNELW
GHDGNDTIRGRGGDDILRGGLGLDTLYGEDGNDIFLQDDETVSDDIDGGAGLDTVDYS
AMIHPGRIVAPHEYGFGIEADLSREWVRKASALGVDYYDNVRNVENVIGTSMKDVLIG
DAQANTLMGQGGDDTVRGGDGDDLLFGGDGNDMLYGDAGNDTLYGGLGDDTLEGG
AGNDWFGQTQAREHDVLRGGDGVDTVDYSQTGAHAGIAAGRIGLGILADLGAGRVDK
LGEAGSSAYDTVSGIENVVGTELADRITGDAQANVLRGAGGADVLAGGEGDDVLLGG
DGDDQLSGDAGRDRLYGEAGDDWFFQDAANAGNLLDGGDGRDTVDFSGPGRGLDAG
AKGVFLSLGKGFASLMDEPETSNVLRNIENAVGSARDDVLIGDAGANVLNGLAGNDVL
SGGAGDDVLLGDEGSDLLSGDAGNDDLFGGQGDDTYLFGVGYGHDTIYESGGGHDTIR
INAGADQLWFARQGNDLEIRILGTDDALTVHDWYRDADHRVEIIHAANQAVDQAGIEK
LVEAMAQYPDPGHHHHHH
SEQ ID NO: 7-Adenylate cyclase plus His tag 3
TENVQYRHVELARVGQLVEVDTLEHVQHIIGGAGNDSITGNAHDNFLAGGSGDDRLDG
GAGNDTLVGGEGQNTVIGGAGDDVFLQDLGVWSNQLDGGAGVDTVKYNVHQPSEER
LERMGDTGIHADLQKGTVEKWPALNLFSVDHVKNIENLHGSRLNDRIAGDDQDNELW
GHDGNDTIRGRGGDDILRGGLGLDTLYGEDGNDIFLQDDETVSDDIDGGAGLDTVDYS
AMIHPGRIVAPHEYGFGIEADLSREWVRKASALGVDYYDNVRNVENVIGTSMKDVLIG
DAQANTLMGQGGDDTVRGGDGDDLLFGGDGNDMLYGDAGNDTLYGGLGDDTLEGG
AGNDWFGQTQAREHDVLRGGDGVDTVDYSQTGAHAGIAAGRIGLGILADLGAGRVDK
LGEAGSSAYDTVSGIENVVGTELADRITGDAQANVLRGAGGADVLAGGEGDDVLLGG
DGDDQLSGDAGRDRLYGEAGDDWFFQDAANAGNLLDGGDGRDTVDFSGPGRGLDAG
AKGVFLSLGKGFASLMDEPETSNVLRNIENAVGSARDDVLIGDAGANVLNGLAGNDVL
SGGAGDDVLLGDEGSDLLSGDAGNDDLFGGQGDDTYLFGVGYGHDTIYESGGGHDTIR
INAGADQLWFARQGNDLEIRILGTDDALTVHDWYRDADHRVEIIHAANQAVDQAGIEK
LVEAMAQYPDPGGHHHHHH
SEQ ID NO: 8-Fragment plus NspA signal peptide
MKKALATLIALALPAAALAEGTENVQYRHVELARVGQLVEVDTLEHVQHIIGGAGNDS
ITGNAHDNFLAGGSGDDRLDGGAGNDTLVGGEGQNTVIGGAGDDVFLQDLGVWSNQL
DGGAGVDTVKYNVHQPSEERLERMGDTGIHADLQKGTVEKWPALNLFSVDHVKNIEN
LHGSRLNDRIAGDDQDNELWGHDGNDTIRGRGGDDILRGGLGLDTLYGEDGNDIFLQD
DETVSDDIDGGAGLDTVDYSAMIHIPGRIVAPHEYGFGIEADLSREWVRKASALGVDYY
DNVRNVENVIGTSMKDVLIGDAQANTLMGQGGDDTVRGGDGDDLLFGGDGNDMLYG
DAGNDTLYGGLGDDTLEGGAGNDWFGQTQAREHDVLRGGDGVDTVDYSQTGAHAGI
AAGRIGLGILADLGAGRVDKLGEAGSSAYDTVSGIENVVGTELADRITGDAQANVLRG
AGGADVLAGGEGDDVLLGGDGDDQLSGDAGRDRLYGEAGDDWFFQDAANAGNLLD
GGDGRDTVDFSGPGRGLDAGAKGVFLSLGKGFASLMDEPETSNVLRNIENAVGSARDN
VLIGDAGANVLNGLAGNDVLSGGAGDDVLLGDEGSDLLSGDAGNDDLFGGQGDDTYL
FGVGYGHDTIYESGGGHDTIRINAGADQLWFARQGNDLEIRILGTDDALTVHDWYRDA
DHRVEIIHAANQAVDQAGIEKLVEAMAQYPDPGGHHHHHH
SEQ ID NO: 9-linker 1
GSGS
SEQ ID NO 10: Linker 2
GSGGGG
SEQ ID NO: 11-linker 3
GSGSGGGG
SEQ ID NO: 12-His tag 1
GGHHHHHH
SEQ ID NO: 13-His tag 2
GHHHHHH
SEQ ID NO: 14-Histag3
HHHHHH
SEQ ID NO: 15-AC domain (from residue 1 to residue 400).
QQSHQAGYANAADRESGIPAAVLDGIKAVAKEKNATLMFRLVNPHSTSLIAEGVATKG
LGVHAKSSDWGLQAGYIPVNPNLSKLFGRAPEVIARADNDVNSSLAHGHTAVDLTLSK
ERLDYLRQAGLVTGMADGVVASNHAGYEQFEFRVKETSDGRYAVQYRRKGGDDFEA
VKVIGNAAGIPLTADIDMFAIMPHLSNFRDSARSSVTSGDSVTDYLARTRRAASEATGGL
DRERIDLLWKIARAGARSAVGTEARRQFRYDGDMNIGVITDFELEVRNALNRRAHAVG
AQDVVQHGTEQNNPFPEADEKIFVVSATGESQMLTRGQLKEYIGQQRGEGYVFYENRA
YGVAGKSLFDDGLGAAPGVPSGRSKFSPDVLETVPASPGLRRPSLGAVERQ
SEQ ID NO: 16-AC domain (from residue 1 to residue 400) with GS insertion
QQSHQAGYANAADRESGIPAAVLDGIKAVAKEKNATLMFRLVNPHSTSLIAEGVATKG
LGVHAKSSDWGLQAGYIPVNPNLSKLFGRAPEVIARADNDVNSSLAHGHTAVDLTLSK
ERLDYLRQAGLVTGMADGVVASNHAGYEQFEFRVKETSDGRYAVQYRRKGGDDFEA
VKVIGNAAGIPLTADGSIDMFAIMPHLSNFRDSARSSVTSGDSVTDYLARTRRAASEATG
GLDRERIDLLWKIARAGARSAVGTEARRQFRYDGDMNIGVITDFELEVRNALNRRAHA
VGAQDVVQHGTEQNNPFPEADEKIFVVSATGESQMLTRGQLKEYIGQQRGEGYVFYEN
RAYGVAGKSLFDDGLGAAPGVPSGRSKFSPDVLETVPASPGLRRPSLGAVERQ
SEQ ID NO: 17-AC domain (from residue 360 to residue 493).
DGLGAAPGVPSGRSKFSPDVLETVPASPGLRRPSLGAVERQDSGYDSLDGVGSRSFSLGE
VSDMAAVEAAELEMTRQVLHAGARQDDAEPGVSGASAHWGQRALQGAQAVAAAQR
LVHAIALMTQFGRAGSTNT
SEQ ID NO: 18-AC domain (from residue 1 to residue 400).
MQQSHQAGYANAADRESGIPAAVLDGIKAVAKEKNATLMFRLVNFHSTSLIAEGVATK
GLGVHAKSSDWGLQAGYIPVNPNLSKLFGRAPEVIARADNDVNSSLAHGHTAVDLTLS
KERLDYLRQAGLVTGMADGVVASNHAGYEQFEFRVKETSDGRYAVQYRRKGGDDFE
AVKVIGNAAGIPLTADIDMFAIMPHLSNFRDSARSSVTSGDSVTDYLARTRRAASEATG
GLDRERIDLLWKIARAGARSAVGTEARRQFRYDGDMNIGVITDFELEVRNALNRRAHA
VGAQDVVQHGTEQNNPFPEADEKIFVVSATGESQMLTRGQLKEYIGQQRGEGYVFYEN
RAYGVAGKSLFDDGLGAAPGVPSGRSKFSPDVLETVPASPGLRRPSLGAVERQ
SEQ ID NO: 19-AC domain (from residue 1 to residue 400) with GS insertion
MQQSHQAGYANAADRESGIPAAVLDGIKAVAKEKNATLMFRLVNPHSTSLIAEGVATK
GLGVHAKSSDWGLQAGYIPVNPNLSKLFGRAPEVIARADNDVNSSLAHGHTAVDLTLS
KERLDYLRQAGLVTGMADGVVASNHAGYEQFEFRVKETSDGRYAVQYRRKGGDDFE
AVKVIGNAAGIPLTADGSIDMFAIMPHLSNFRDSARSSVTSGDSVTDYLARTRRAASEAT
GGLDRERIDLLWKIARAGARSAVGTEARRQFRYDGDMNIGVITDFELEVRNANNRRAH
AVGAQDVVQHGTEQNNPFPEADEKIFVVSATGESQMLTRGQLKEYIGQQRGEGYVFYE
NRAYGVAGKSLFDDGLGAAPGVPSGRSKFSPDVLETVPASPGLRRPSLGAVERQ
SEQ ID NO: 20-AC domain (from residue 360 to residue 493).
MDGLCAAPGVPSGRSKFSPDVLETVPASPGLRRPSLGAVERQDSCYDSLDGVGSRSFSL
GEVSDMAAVEAAEEEMTRQVEHAGARQDDAEPGVSGASAHWGQRAEQGAQAVAAA
QRLVHAIALMTQFGRAGSTNT
SEQ ID NO: 21-AC domain (from residue 1 to residue 400). Cytoplasmic
expression of his-tagged (composed of GGHHHHHH sequence) domain
MQQSHQAGYANAADRESGIPAAVLDGIKAVAKEKNATLMFRLVNPHSTSLIAEGVATK
GLGVHAKSSDWGLQAGYIPVNPNLSKLFGRAPEVIARADNDVNSSLAHGHTAVDLTLS
KERLDYLRQAGLVTGMADGVVASNHAGYEQFEFRVKETSDGRYAVQYRRKGGDDFE
AVKVIGNAAGIPLTADgsIDMFAIMPHLSNFRDSARSSVTSGDSVTDYLARTRRAASEAT
GGLDRERIDLLWKIARAGARSAVGTEARRQFRYDGDMNIGVITDFELEVRNALNRRAH
AVGAQDVVQHGTEQNNPFPEADEKIFVVSATGESQMLTRGQFKEYIGQQRGEGYVFYE
NRAYGVAGKSLFDDGLGAAPGVPSGRSKFSPDVLETVPASPGLRRPSLGAVERQGGHH
HHHH
SEQ ID NO: 22-AC domain (from residue 360 to residue 493). Cytoplasmic
expression of his-tagged (composed of GGHHHHHH sequence) domain.
MDGLGAAPGVPSGRSKFSPDVLETVPASPGLRRPSLGAVERQDSGYDSLDGVGSRSFSL
GEVSDMAAVEAAELEMTRQVLHAGARQDDAEPGVSGASAHWGQRALQGAQAVAAA
QRLVHAIALMTQFGRAGSTNTGGHHHHHH
SEQ ID NO: 23-Adenylate cyclase fragment without His tag
MTENVQYRHVELARVGQLVEVDTLEHVQHIIGGAGNDSITGNAHDNFLAGGSGDDRLD
GGAGNDTLVGGEGQNTVIGGAGDDVFLQDLGVWSNQLDGGAGVDTVKYNVHQPSEE
RLERMGDTGIHADLQKGTVEKWPALNLFSVDHVKNIENLHGSRLNDRIAGDDQDNEL
WGHDGNDTIRGRGGDDILRGGLGLDTLYGEDGNDIFLQDDETVSDDIDGGAGLDTVDY
SAMIHPGRIVAPHEYGFGIEADLSREWVRKASALGVDYYDNVRNVENVIGTSMKDVLIG
DAQANTLMGQGGDDTVRGGDGDDLLFGGDGNDMLYGDAGNDTLYGGLGDDTLEGG
AGNDWFGQTQAREHDVLRGGDGVDTVDYSQTGAHAGIAAGRIGLGILADLGAGRVDK
LGEAGSSAYDTVSGIENVVGTELADRITGDAQANVLRGAGGADVLAGGEGDDVLLGG
DGDDQLSGDAGRDRLYGEAGDDWFFQDAANAGNLLDGGDGRDTVDFSGPGRGLDAG
AKGVFLSLGKGFASLMDEPETSNVLRNIENAVGSARDDVLIGDAGANVLNGLAGNDVL
SGGAGDDVLLGDEGSDLLSGDAGNDDLFGGQGDDTYLFGVGYGHDTIYESGGGHDTIR
INAGADQLWFARQGNDLEIRILGTDDALTVHDWYRDADHRVEIIHAANQAVDQAGIEK
LVEAMAQYPDP

[1] Needleman & Wunsch (1970) J. Mol. Biol. 48, 443-453.

[2] Rice et al. (2000) Trends Genet 16:276-277.

[3] U.S. Pat. No. 5,707,829

[4] Vaccine Design (1995) eds. Powell & Newman. ISBN: 030644867X. Plenum.

[5] WO90/14837.

[6] U.S. Pat.No. 5,057,540.

[7] WO96/33739.

[8] EP-A-0109942.

[9] WO96/11711.

[10] WO00/07621.

[11] Barr et al. (1998) Advanced Drug Delivery Reviews 32:247-271.

[12] Sjolanderet et al. (1998) Advanced Drug Delivery Reviews 32:321-338.

[13] EP-A-0689454.

[14] Johnson et al. (1999) Bioorg Med Chem Lett 9:2273-2278.

[15] Evans et al. (2003) Expert Rev Vaccines 2:219-229.

[16] Meraldi et al. (2003) Vaccine 21:2485-2491.

[17] Pajak et al. (2003) Vaccine 21:836-842.

[18] U.S. Pat. No. 6,090,406.

[19] U.S. Pat. No. 5,916,588.

[20] EP-A-0626169.

[21] WO99/11241.

[22] WO94/00153.

[23] WO98/57659.

[24] European patent applications 0835318, 0735898 and 0761231.

[25] Vaccines. (eds. Plotkin & Orenstein). 4th edition, 2004, ISBN: 0-7216-9688-0.

[26] Rappuoli et al. (1991) TIBTECH 9:232-238.

[27] Gennaro (2000) Remington: The Science and Practice of Pharmacy. 20th edition, ISBN: 0683306472.

[28] Methods In Enzymology (S. Colowick and N. Kaplan, eds., Academic Press, Inc.)

[29] Handbook of Experimental Immunology, Vols. I-IV (D. M. Weir and C. C. Blackwell, eds, 1986, Blackwell Scientific Publications)

[30] Sambrook et al. (2001) Molecular Cloning: A Laboratory Manual, 3rd edition (Cold Spring Harbor Laboratory Press).

[31] Handbook of Surface and Colloidal Chemistry (Birdi, K. S. ed., CRC Press, 1997)

[32] Ausubel et al. (eds) (2002) Short protocols in molecular biology, 5th edition (Current Protocols).

[33] Molecular Biology Techniques: An Intensive Laboratory Course, (Ream et al., eds., 1998, Academic Press)

[34] PCR (Introduction to Biotechniques Series), 2nd ed. (Newton & Graham eds., 1997, Springer Verlag)

[35] Geysen et al. (1984) PNAS USA 81:3998-4002.

[36] Carter (1994) Methods Mol Biol 36:207-23.

[37] Jameson, B A et al. 1988, CABIOS 4(1):181-186.

[38] Raddrizzani & Hammer (2000) Brief Bioinform 1(2):179-89.

[39] Bublil et al. (2007) Proteins 68(1):294-304.

[40] De Lalla et al. (1999) J. Immunol. 163:1725-29.

[41] Kwok et al. (2001) Trends Immunol 22:583-88.

[42] Brusic et al. (1998) Bioinformatics 14(2):121-30

[43] Meister et al. (1995) Vaccine 13(6):581-91.

[44] Roberts et al. (1996) AIDS Res Hum Retroviruses 12(7):593-610.

[45] Maksyutov & Zagrebelnaya (1993) Comput Appl Biosci 9(3):291-7.

[46] Feller & de la Cruz (1991) Nature 349(6311):720-1.

[47] Hopp (1993) Peptide Research 6:183-190.

[48] Welling et al. (1985) FEBS Lett. 188:215-218.

[49] Davenport et al. (1995) Immunogenetics 42:392-297.

[50] Tsurui & Takahashi (2007) J Pharmacol Sci. 105(4):299-316.

[51] Tong et al. (2007) Brief Bioinform. 8(2):96-108.

[52] Schirle et al. (2001) J Immunol Methods. 257(1-2):1-16.

[53] Chen et al. (2007) Amino Acids 33(3):423-8.

[54] Current Protocols in Molecular Biology (F. M. Ausubel et al., eds., 1987) Supplement 30

[55] Smith & Waterman (1981) Adv. Appl. Math. 2: 482-489.

1. A polypeptide comprising an amino acid sequence:
A-X-B
wherein: ‘X’ is an amino acid sequence consisting of a sequence having at least 99% identity with SEQ ID NO: 2, 3 or 4; ‘A’ is an optional N terminal amino acid sequence; ‘B’ is an optional C terminal amino acid sequence, and wherein ‘A’ and ‘B’ are not derived from adenylate cyclase or a fragment thereof.

2. The polypeptide of claim 1 wherein ‘A’ is an N terminal methionine residue, ‘X’ is an amino acid sequence selected from the group consisting of SEQ ID NO: 2, 3 and 4 and wherein ‘B’ is absent.

3. The polypeptide of claim 1 which consists of a sequence having 99% identity with SEQ ID NO: 23.

4. The polypeptide of claim 1 wherein ‘A’ and/or ‘B’ is a histidine tag.

5. The polypeptide of claim 4 which consists of a sequence having at least 99% identity with SEQ ID NO: 5, 6, 7 or 8.

6. The polypeptide of claim 5 which consists of a sequence having 100% identity with SEQ ID NO: 5, 6, 7 or 8.

7. The polypeptide of claim 1 capable of eliciting an antibody response comprising antibodies that bind to the Adenylate cyclase protein having amino acid sequence of SEQ ID NO:1, for example, as measured by adenylate cyclase toxin neutralisation assay.

8. A nucleic acid encoding a polypeptide according to claim 1.

9. A bacterium that comprises the nucleic acid of claim 8.

10. An immunogenic composition comprising a polypeptide according to claim 1 or a nucleic acid according to claim 8.

11. The immunogenic composition according to claim 10 which comprises an adjuvant.

12. The immunogenic composition of claim 10 which comprises a divalent metal salt.

13. The immunogenic composition of claim 12 wherein the divalent metal salt is a Calcium salt, for example, Calcium chloride.

14. A therapeutic method comprising:
administering the immunogenic composition according to claim 10 to a subject.

15. A method for treating or preventing disease and/or infection caused by Bordetella pertussis, comprising:
administering an effective amount of the immunogenic composition according to claim 10 to a subject.

16. A method or treating or preventing disease and/or infection caused by Bordetella pertussis in a mammal, comprising:
administering an effective amount of the polypeptide according to claim 1 or a nucleic acid according to claim 8 to said mammal.

17. The method according to claim 16, wherein said mammal is a human.
